SEQUENCE LISTING (4 sequences)

SEQ ID NO: 1 - protein, 9 aa, Homo sapiens
AAGIGILTV

SEQ ID NO: 2 - protein, 10 aa, Homo sapiens
EAAGIGILTV

SEQ ID NO: 3 - protein, 10 aa, Artificial sequence
  Tracer peptide
FLPSDCFPSV

SEQ ID NO: 4 - protein, 10 aa, Artificial sequence
  Mutated peptide
ELAGIGILTV

(19) United States
(12) Patent Application Publication
(10) Pub. No.: US 2005/0095655 A1
(43) Pub. Date: May 5, 2005

(54) Solution-based methods for detecting MHC-binding peptides

(76) Inventors: Felix A. Montero-Julian, Marseille (FR); Sylvain Monseaux, Marseille (FR); Antje Necker, La Ciotat (FR)

Correspondence Address: BECKMAN COULTER, INC.;C/O DLA PIPER RUDNICK GRAY CARY US LLP, 4365 EXECUTIVE DR, SUITE 1100, SAN DIEGO, CA, 92121-2133, US

(21) Appl. No.: 10/782,664

(22) Filed: Feb. 18, 2004

Related U.S. Application Data
(60) Provisional application No. 60/517,019, filed on Nov. 3, 2003.

(51) Int. Cl. G01N 33/53; G01N 33/567

(52) U.S. Cl. 435/7.2

(57) ABSTRACT

Solution-based methods for identifying an MHC-binding peptide or measuring affinity of MHC-binding peptides for an MHC monomer, or modified MHC monomer by incubating at least one MHC monomer or modified MHC monomer having a bound template MHC-binding peptide, an excess amount of a competitor peptide, and a tracer MHC-binding peptide tagged with a detectable label so as to allow competition binding between the three peptides At least a portion of the competitor peptide exchanges with the template peptide and a difference in signal produced by the detectable label in the total sample as compared with signal produced solely by monomers after the competition assay is obtained and used to calculate affinity of the competitor peptide for the monomer. These methods are useful in peptide discovery programs and exchanged monomers can be further tested for activity in tetramer cell staining assays.

This application claims the benefit of priority under 35 U.S.C. § 119 of U.S. Ser. No. 60/517,019, filed Nov. 3, 2003, the entire contents of which are incorporated herein by reference.

### BACKGROUND OF THE INVENTION

1. Field of the Invention

The invention relates generally to the field of immunoassays, especially using immunoassays to detect and measure binding of peptides to MHC alleles.

2. Background Information

The Class I histocompatibility ternary complex consists of three parts associated by noncovalent bonds. A transmembrane protein, called the MHC heavy chain is mostly exposed at the cell surface. The cell surface domains of the MHC heavy chain contain two segments of alpha helix that form two ridges with a groove between them. A short peptide binds noncovalently (“fits”) into this groove between the two alpha helices, and a molecule of beta-2 microglobulin binds noncovalently along side the outer two domains of the MHC monomer, forming a ternary complex. Peptides that bind noncovalently to one MHC subtype heavy chain usually will not bind to another subtype. However, all bind with the same type of beta-2 microglobulin. MHC molecules are synthesized and displayed by most of the cells of the body.

In humans, MHC molecules are referred to as HLA molecules. Humans primarily synthesize three different sub-types of MHC class I molecules designated HLA-A, HLA-B and HLA-C, differing only in the heavy chains.

The MHC works coordinately with a specialized type of T cell (the cytotoxic T cell) to rid the body of “nonself” or foreign viral proteins. The antigen receptor on T-cells recognizes an epitope that is a mosaic of the bound peptide and portions of the alpha helices making up the groove flanking it. Following generation of peptide fragments by cleavage of a foreign protein, the presentation of peptide fragments by the MHC molecule allows for MHC-restricted cytotoxic T cells to survey cells for the expression of “nonself” or foreign viral proteins. A functional T-cell will exhibit a cytotoxic immune response upon recognition of an MHC molecule containing bound antigenic peptide for which the T-cell is specific.

In the performance of these functions in humans, HLA-A, B, and C heavy chains interact with a multitude of peptides of about 8 to about 10, possibly about 8 to about 11, or about 8 to about 12 amino acids in length. Only certain peptides bind into the binding pocket in the heavy chain of each HLA sub-type as the monomer folds, although certain subtypes cross-react. By 1995, complete coding region sequences had been determined for each of 43 HLA-A, 89 HLA-B and 11 HLA-C alleles (P. Parham et al., Immunology Review 143:141-180, 1995).

Class II histocompatibility molecules consist of two transmembrane polypeptides that interact to form a groove at their outer end which, like the groove in class I molecules, non-covalently associates with an antigenic peptide. However, the antigenic peptides bound to class II molecules are derived from antigens that the cell has taken in from its surroundings. In addition, peptides that bind to class II histocompatibility molecules are 15 to about 25 or to about 30 amino acids in length. Only cells, such as macrophages, dendritic cells and B lymphocytes, that specialize in taking up antigen from extracellular fluids, express class II molecules.

It has long been thought that discovery of which antigen fragments will be recognized by class I MHC-restricted T-cells can lead to development of effective vaccines against cancer and viral infections. A number of approaches have been developed wherein algorithms are used to predict the amino acid sequence of HLA A, B, or C-binding peptides and several are available on the internet. For example, U.S. Pat. No. 6,037,135 describes a matrix-based algorithm that ranks peptides for likelihood of binding to any given HLA-A allele. Similarly, most prediction methods are limited to a set of alleles. Consequently, the predicted peptides may not bind to MHC monomers from a whole population of patients and thus may not be globally effective.

Another approach to identifying MHC-binding peptides uses a competition-based binding assay. All competition assays yield a comparison of binding affinities of different peptides. However, such assays do not yield an absolute dissociation constant since the result is dependent on the reference peptide used.

Still another approach used for determining MHC-binding peptides is the classical reconstitution assay, e.g. using “T2” cells, in which cells expressing an appropriate MHC allele are “stripped” of a native binding peptide by incubating at pH 2-3 for a short period of time. Then, to determine the binding affinity of a putative MHC-binding peptide for the same MHC allele, the stripped MHC monomer is combined in solution with the putative MHC-binding peptide, beta-2-microglobulin and a conformation-dependent monoclonal antibody. The difference in fluorescence intensity determined between cells incubated with and without the test binding peptide after labeling, for example, either directly with the labeled monoclonal antibody or a fluorescence-labeled secondary antibody, can be used to determine binding of the test peptide. However, soluble MHC monomers stripped at low pH aggregate immediately, making their use in high through-put assays difficult and impractical.

There are currently a series of in vitro assays for cell mediated immunity which use cells from the donor. The assays include situations where the cells are from the donor, however, many assays provide a source of antigen presenting cells from other sources, e.g., B cell lines. These in vitro assays include the cytotoxic T lymphocyte assay; lymphoproliferative assays, e.g., tritiated thymidine incorporation; the protein kinase assays, the ion transport assay and the lymphocyte migration inhibition function assay (Hickling, J. K. et al, J. Virol., 61: 3463 (1987); Hengel, H. et al, J. Immunol., 139: 4196 (1987); Thorley-Lawson, D. A. et al, Proc. Natl. Acad. Sci. USA, 84: 5384 (1987); Kadival, G. J. et al, J. Immunol., 139: 2447 (1987); Samuelson, L. E. et al, J. Immunol., 139: 2708 (1987); Cason, J. et al, J. Immunol. Meth., 102: 109 (1987); and Tsein, R. J. et al, Nature, 293: 68 (1982)). These assays are disadvantageous in that they may lack true specificity for cell mediated immunity activity, they require antigen processing and presentation by an APC of the same MHC type, they are slow (sometimes lasting several days), and some are subjective and/or require the use of radioisotopes.

Yet another approach to identifying MHC class I-binding peptides utilizes formation of MHC tetramers, which are complexes of four MHC monomers with streptavidin, a molecule having tetrameric binding sites for biotin, to which is bound a fluorochrome, e.g., phycoerythrin (PE). For class I monomers, soluble subunits of β-microglobulin, the peptide fragment containing a putative T-cell epitope, and an MHC heavy chain corresponding to the predicted MHC subtype of the peptide fragment of interest, are obtained by expression of the polypeptides in host cells. Each of the four monomers contained in the MHC tetramer is produced as a monomer by refolding these soluble subunits under conditions that favor assembly of the soluble units into reconstituted monomers, each containing a beta-2-microglobulin, a peptide fragment, and the corresponding MHC heavy chain. An MHC tetramer is constructed from the monomers by biotinylation of the monomers and subsequent contact of the biotinylated reconstituted monomers with fluorochrome-labeled streptavidin. When contacted with a diverse population of T cells, such as is contained in a sample of the peripheral blood lymphocytes (PBLs) of a subject, those tetramers containing monomers that are recognized by a T cell in the sample will bind to the matched T cell. Contents of the reaction is analyzed using fluorescence flow cytometry, to determine, quantify and/or isolate those T-cells having an MHC tetramer bound thereto (See U.S. Pat. No. 5,635,363). Since the tetramer contains a fluorescent moiety, T cells having a bound tetramer are said to be “stained.”

At least one other test is required to determine whether a test peptide recognized by a T-cell by the MHC tetramer assay will activate the T-cell to generate an immune response, a so-called “functional test”. The enzyme-linked immunospot (ELISpot) assay has been adapted for the detection of individual cells secreting specific cytokines or other effector molecules by attachment of a monoclonal antibody specific for a cytokine or effector molecule on a microplate. Cells stimulated by an antigen are contacted with the immobilized antibody. After washing away cells and any unbound substances, a tagged polyclonal antibody or more often, a monoclonal antibody, specific for the same cytokine or other effector molecule is added to the wells. Following a wash, a colorant that binds to the tagged antibody is added such that a blue-black colored precipitate (or spot) forms at the sites of cytokine localization. The spots can be counted manually or with automated ELISpot reader system to quantitated the response. A final confirmation of T-cell activation by the test peptide may require in vivo testing, for example in a mouse model. Thus, the route to final confirmation of the efficacy of an MHC-binding peptide is expensive and time consuming.

It has also been observed that exogenous peptides can bind to immunopurified HLA molecules. Chen and Parham demonstrated in 1989 (Nature, 337: 743-745) by gel filtration chromatography that influenza matrix peptides and influenza nucleoprotein peptides bind selectively to affinity-purified preparations of detergent solubilized HLA-A2 AND HLA-B37, respectively. Later, it was reported that some cell lines like RMA-S cells express, at the cell surface, empty HLA molecules that can be stabilized by adding exogenous peptides.

Smith et al. (1992 Proc. Natl. Acad. Sci. USA. 89: 7767-7771) reported peptide exchange using Ld-transfected L-cells, a fibroblastic cell line, and concluded that the peptide exchange of radiolabeled CMV peptide occurs at the cell surface. At the cell surface Ld class I molecules were shown to be readily capable of peptide exchange, with the amount of peptide exchange being governed by the number of Ld molecules and by the dissociation rate of the bound peptide. It was concluded that cell surface peptide ligand exchange by H-2 Ld is a generalizable phenomenon.

Later, Hörig et al. (1997 Proc. Natl. Acad. Sci. USA. 94: 13826-13831) studied the exchange of peptide and beta-2m using purified, recombinant H-2 Kb/peptide complexes in a cell-free in vitro system and concluded that the exchange of competitor peptide was primarily dependent on the off-rate of the original peptide in the class I binding groove, that peptide exchange was not enhanced by the presence of exogenous beta-2 m, as believed by others, and that peptides could be exchanged into class I molecules over a pH range of 5.5 to 7.5, conditions prevalent in certain endocytic compartments.

However, there is still a need in the art for new and better systems and methods for preliminary screening assays identifying putative MHC class I-binding peptides and for measuring peptide binding to MHC class I alleles, such as HLA-A, B or C, especially an in vitro solution-based format. There is also a need in the art to develop methods to determine the MHC-binding affinity of MHC-binding peptides in solution.

### SUMMARY OF THE INVENTION

This invention is based on the discovery that a solution-based competition peptide exchange assay can be used to rapidly compare and quantify the binding affinity of peptides of unknown binding properties for MHC heavy chain monomers and modified MHC monomers. Moreover, using a third labeled peptide of known affinity in a competition solution-based assay, the exchange reaction can be measured by observing the degree to which the labeled peptide out-competes the test peptide. It is the discovery of the present invention that such binding can be utilized in a solution-based competition peptide exchange assay to rapidly compare and quantify the binding affinity of peptides of unknown binding properties for MHC heavy chain monomers and modified MHC monomers.

Accordingly, in one embodiment the invention provides methods for identifying an MHC-binding peptide for an MHC monomer, or modified MHC monomer by incubating under suitable liquid phase conditions a sample containing at least one MHC monomer or modified MHC monomer having bound thereto a template MHC-binding peptide, an excess amount of a first competitor peptide, and a tracer MHC-binding peptide tagged with a detectable label. The template peptide is selected to have lower or intermediate affinity as compared with the tracer peptide for the monomer. In solution, the first competitor peptide, the template peptide, and the tracer peptide compete for binding to the MHC monomer or modified MHC monomer. Readings of signal from the detectable label taken from the total sample and produced by monomer obtained from the sample after the incubation are compared to determine a difference, wherein the difference indicates that the first competitor peptide is an MHC-binding peptide for the monomer.

In another embodiment, the invention provides methods for measuring affinity of MHC-binding peptides for an MHC monomer, or modified MHC monomer by incubating under suitable liquid phase conditions a sample containing: at least one MHC monomer or modified MHC monomer having bound thereto a template MHC-binding peptide, a molar excess amount of a first competitor peptide, and a tracer MHC-binding peptide tagged with a detectable label. The template peptide has lower affinity than the tracer peptide for the monomer. After competition between the first competitor peptide, the template peptide, and the tracer peptide for binding to the MHC monomer or modified MHC monomer, at least a portion of the first competitor peptide exchanges with the template peptide. The difference in signal produced by the detectable label in the total sample as compared with signal produced solely by monomer obtained from the sample after the incubation indicates affinity of the first competitor peptide for the monomer.

In yet another embodiment, the invention provides methods for measuring function of an MHC-monomer or modified MHC monomer bound to an exchanged peptide for binding to a cell displaying a peptide-restricted T-cell receptor (TCR). This peptide functionality assay is conducted by incubating together under suitable liquid phase conditions a sample comprising: MHC monomers or modified MHC monomers having bound thereto a template MHC-binding peptide, an excess amount of a first competitor peptide, and a tracer MHC-binding peptide tagged with a first detectable label so as to allow competition between the first competitor peptide, the template peptide and the tracer peptide for binding to the MHC monomer or modified MHC monomer, wherein the template peptide has lower affinity than the tracer peptide for the monomer. In at least a portion of the monomers, the first competitor peptide exchanges with the template peptide to form exchanged monomers. A multimer of the exchanged monomers is formed by binding the exchanged monomers with a multivalent entity labeled with a second detectable label. Binding of the exchanged monomers in the multimer with the TCR of the cell is then determined, wherein the binding indicates the first competitor peptide in the exchanged monomers is specific for the TCR.

In still another embodiment, the invention provides systems useful for identifying an MHC-binding peptide for an MHC monomer, or modified MHC monomer. The invention systems include at least one MHC monomer or modified MHC monomer having bound thereto a template MHC-binding peptide, and a tracer MHC-binding peptide tagged with a detectable label, wherein the template peptide has lower affinity than the tracer peptide for the monomer. The invention system may further include an instruction for using the system.

### BRIEF DESCRIPTION OF THE FIGURES

FIG. 1A is an illustration of the invention solution-based peptide exchange.

FIG. 1B is a flow chart illustrate the procedure used in the invention solution-based peptide exchange.

FIG. 2 is an illustration of a scale for determining the degree of affinity of an HLA peptide binder.

FIG. 3 is a graph showing binding of monomer HLA-A*0201 (A245V)/Mart-1 27-35 exchanged with HBVc-FITC and HIVpol-FITC peptides.

FIG. 4 is a graph of the chromatograph profile of the exchanged monomers of FIG. 4.

FIGS. 5A-D are graphs showing exchanges of different monomers with different concentrations of the FITC peptides HBc and HIVpol. FIG. 6A shows Monomer HLA-A*0201/CMVpp65; FIG. 6B shows monomer HLA-A*0201/HIVpol; FIG. 6C shows monomer HLA-A*0201/Mart-1 27-35 and FIG. 6D shows monomer HLA-A*0201/Mart-1 2635.

FIG. 6A is a graph showing the dose response curve of monomer HBVc-FITC.

FIG. 6B is a graph showing the % of B/T as a function of the concentration for the monomer of FIG. 7A.

FIG. 7 is a schematic representation of the B1G6 assay.

FIG. 8 is a graph of the dose response curve of the antibody of the B1G6 assay in presence of different concentrations of monomer.

FIG. 9 is a graph showing the correlation between the calculated monomer concentration determined using two different monomers as standards.

FIG. 10 is a graph showing the standard curve obtained using B1G6-PE mAb and monomer HLA-A*0201/HBVc-FITC.

FIG. 11 staining of cell lines with monomers containing test peptides when tetramerized with the SA-PE alone and their respective positive control

FIG. 12 shows the effect of various amounts of excess peptide on staining of Jurkat cells with exchanged tetramers and control tetramers.

FIG. 13 shows the effect of various amounts of excess peptide on staining of RBL 80210 cells with exchanged tetramers and control tetramers.

FIG. 14A shows the contribution of the tracer peptide to stabilization of the peptide and effect on results obtained by flow cytometry from staining of RBL 80210 cells with exchanged Mart1 26-35 monomer with HIV/Pol peptide.

FIG. 14B shows the contribution of the tracer peptide to stabilization of the peptide and effect on results obtained by flow cytometry from staining of Jurkat cells with exchanged Mart1 26-35 monomer with HIV/Pol peptide.

FIG. 15 is a graph showing the correlation between the % of the exchange and the % of the control obtained by flow cytometry.

### DETAILED DESCRIPTION OF THE INVENTION

As used herein, the terms “MHC monomer” and “HLA monomer” refer to a class I MHC heavy chain that maintains the ability to assemble or is assembled into a ternary complex with an appropriate MHC-binding or HLA-binding peptide and beta-2 microglobulin (beta-2m) under renaturing conditions. The terms “MHC monomer” and HLA monomer” are also used to refer to the denatured form of the monomer that results from subjecting the ternary complex to denaturing conditions, causing the monomer to unfold and dissociate from an MHC-binding peptide and from beta-2 microglobulin.

As used herein, the terms “modified MHC monomer” and “modified HLA monomer” refer to class I monomers as described above, but which have been engineered to introduce modifications as described below. These terms also encompass functional fragments of the MHC monomer that maintain the ability to assemble into a ternary complex with an appropriate MHC-binding or HLA-binding peptide and beta-2 microglobulin under renaturing conditions and to dissociate under denaturing conditions. For example, a functional fragment can comprise only the α₁, α₂, α₃, domains, or only α₁, α₂ domains, of the class I heavy chain, i.e., the cell surface domains, that participate in formation of the ternary complex. In another embodiment, modified MHC monomers can be class I heavy chain molecules, or functional fragments thereof, contained in a fusion protein or “single chain” molecule and may further include an amino acid sequence functioning as a linker between cell surface domains of the monomer, a detectable marker or as a ligand to attach the molecule to a solid support that is coated with a second ligand with which the ligand in the fusion protein reacts. Moreover the terms “modified MHC monomer” and “modified HLA monomer” are intended to encompass chimera containing domains of class I heavy chain molecules from more than one species or from more than one class I subclass. For example, a chimera can be prepared by substitution of a mouse beta-2m for human beta-2m in a human HLA-A2 monomer.

Preparation of Monomers

The Class I MHC in humans is located on chromosome 6 and has three loci, HLA-, HLA-B, and HLA-C. The first two loci have a large number of alleles encoding alloantigens. These are found to consist of a 44 Kd heavy chain subunit and a 12 Kd beta-2-microglobulin subunit which is common to all antigenic specificities. For example, soluble HLA-A2 can be purified after papain digestion of plasma membranes from the homozygous human lymphoblastoid cell line J-Y as described by Turner, M. J. et al., J. Biol. Chem. (1977) 252:7555-7567. Papain cleaves the 44 Kd heavy chain close to the transmembrane region, yielding a molecule comprised of α₁, α₂, α₃ domains and beta-2 microglobulin.

The MHC monomers can be isolated from appropriate cells or can be recombinantly produced, for example as described by Paul et al, Fundamental Immunology, 2d Ed., W. E. Paul, ed., Ravens Press N.Y. 1989, Chapters 16-18) and readily modified, as described below.

The term “isolated” as applied to MHC monomers herein refers to an MHC glycoprotein heavy chain of MHC class I, which is in other than its native state, for example, not associated with the cell membrane of a cell that normally expresses MHC. This term embraces a full length subunit chain, as well as a functional fragment of the MHC monomer. A functional fragment is one comprising an antigen binding site and sequences necessary for recognition by the appropriate T cell receptor. It typically comprises at least about 60-80%, typically 90-95% of the sequence of the full-length chain. As described herein, the “isolated” MHC subunit component may be recombinantly produced or solubilized from the appropriate cell source.

It is well known that native forms of “mature” MHC glycoprotein monomers will vary somewhat in length because of deletions, substitutions, and insertions or additions of one or more amino acids in the sequences. Thus, MHC monomers are subject to substantial natural modification, yet are still capable of retaining their functions. Modified protein chains can also be readily designed and manufactured utilizing various recombinant DNA techniques well known to those skilled in the art and described in detail, below. For example, the chains can vary from the naturally occurring sequence at the primary structure level by amino acid substitutions, additions, deletions, and the like. These modifications can be used in a number of combinations to produce the final modified protein chain.

In general, modifications of the genes encoding the MHC monomer may be readily accomplished by a variety of well-known techniques, such as site-directed mutagenesis. The effect of any particular modification can be evaluated by routine screening in a suitable assay for the desired characteristic. For instance, a change in the immunological character of the subunit can be detected by competitive immunoassay with an appropriate antibody. The effect of a modification on the ability of the monomer to activate T cells can be tested using standard in vitro cellular assays or the methods described in the example section, below. Modifications of other properties such as redox or thermal stability, hydrophobicity, susceptibility to proteolysis, or the tendency to aggregate are all assayed according to standard techniques.

This invention provides amino acid sequence modification of MHC monomers prepared with various objectives in mind, including increasing the affinity of the subunit for antigenic peptides and/or T cell receptors, facilitating the stability, purification and preparation of the subunits. The monomers may also be modified to modify plasma half life, improve therapeutic efficacy, or to lessen the severity or occurrence of side effects during therapeutic use of complexes of the present invention. The amino acid sequence modifications of the subunits are usually predetermined variants not found in nature or naturally occurring alleles. The variants typically exhibit the same biological activity (for example, MHC-peptide binding) as the naturally occurring analogue.

Insertional modifications of the present invention are those in which one or more amino acid residues are introduced into a predetermined site in the MHC monomer and which displace the preexisting residues. For instance, insertional modifications can be fusions of heterologous proteins or polypeptides to the amino or carboxyl terminus of the subunits.

Other modifications, include fusions of the monomer with a heterologous signal sequence and fusions of the monomer to polypeptides having enhanced plasma half life (ordinarily>about 20 hours) such as immunoglobulin chains or fragments thereof as is known in the art.

Substitutional modifications are those in which at least one residue has been removed and a different residue inserted in its place. Nonnatural amino acid (i.e., amino acids not normally found in native proteins), as well as isosteric analogs (amino acid or otherwise) are also suitable for use in this invention.

Substantial changes in function or immunological identity are made by selecting substituting residues that differ in their effect on maintaining the structure of the polypeptide backbone (e.g., as a sheet or helical conformation), the charge or hydrophobicity of the molecule at the target site, or the bulk of the side chain. The substitutions which in general are expected to produce the greatest changes in function will be those in which (a) a hydrophilic residue, e.g., serine or threonine, is substituted for (or by) a hydrophobic residue, e.g. leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamine or aspartine; or (d) a residue having a bulky side chain, e.g., phenylalanine, is substituted for (or by) one not having a side chain, e.g., glycine.

Substitutional modifications of the monomers also include those where functionally homologous (having at least about 70% homology) domains of other proteins are substituted by routine methods for one or more of the MHC subunit domains. Particularly preferred proteins for this purpose are domains from other species, such as murine species.

Another class of modifications are deletional modifications. Deletions are characterized by the removal of one or more amino acid residues from the MHC monomer sequence. Typically, the transmembrane and cytoplasmic domains are deleted. Deletions of cysteine or other labile residues also may be desirable, for example in increasing the oxidative stability of the MHC complex. Deletion or substitution of potential proteolysis sites, e.g., ArgArg, is accomplished by deleting one of the basic residues or substituting one by glutaminyl or histidyl residues.

A preferred class of substitutional or deletional modifications comprises those involving the transmembrane region of the subunit. Transmembrane regions of MHC monomers are highly hydrophobic or lipophilic domains that are the proper size to span the lipid bilayer of the cellular membrane. They are believed to anchor the MHC molecule in the cell membrane. Inactivation of the transmembrane domain, typically by deletion or substitution of transmembrane domain hydroxylation residues, will facilitate recovery and formulation by reducing its cellular or membrane lipid affinity and improving its aqueous solubility. Alternatively, the transmembrane and cytoplasmic domains can be deleted to avoid the introduction of potentially immunogenic epitopes. Inactivation of the membrane binding function is accomplished by deletion of sufficient residues to produce a substantially hydrophilic hydropathy profile at this site or by substitution with heterologous residues which accomplish the same result.

A principal advantage of the transmembrane-inactivated MHC monomer is that it may be secreted into the culture medium of recombinant hosts. This variant is soluble in body fluids such as blood and does not have an appreciable affinity for cell membrane lipids, thus considerably simplifying its recovery from recombinant cell culture. Typically, modified MHC monomers of this invention will not have a functional transmembrane domain and preferably will not have a functional cytoplasmic sequence. Such modified MHC monomers will consist essentially of the effective portion of the extracellular domain of the MHC monomer. In some circumstances, the monomer comprises sequences from the transmembrane region (up to about 10 amino acids), so long as solubility is not significantly affected.

For example, the transmembrane domain may be substituted by any amino acid sequence, e.g., a random or predetermined sequence of about 5 to 50 serine, threonine, lysine, arginine, glutamine, aspartic acid and like hydrophilic residues, which altogether exhibit a hydrophilic hydropathy profile. Like the deletional (truncated) monomer, these monomers are secreted into the culture medium of recombinant hosts.

Glycosylation variants are included within the scope of this invention. They include variants completely lacking in glycosylation (unglycosylated) and variants having at least one less glycosylated site than the native form (deglycosylated) as well as variants in which the glycosylation has been changed. Included are deglycosylated and unglycosylated amino acid sequence variants, deglycosylated and unglycosylated subunits having the native, unmodified amino acid sequence. For example, substitutional or deletional mutagenesis is employed to eliminate the N- or O-linked glycosylation sites of the subunit, e.g., the asparagine residue is deleted or substituted for by another basic residue such as lysine or histidine. Alternatively, flanking residues making up the glycosylation site are substituted or deleted, even though the asparagine residues remain unchanged, in order to prevent glycosylation by eliminating the glycosylation recognition site. Additionally, unglycosylated MHC monomers which have the amino acid sequence of the native monomers are produced in recombinant prokaryotic cell culture because prokaryotes are incapable of introducing glycosylation into polypeptides.

Glycosylation variants are conveniently produced by selecting appropriate host cells or by in vitro methods. Yeast, for example, introduce glycosylation which varies significantly from that of mammalian systems. Similarly, mammalian cells having a different species (e.g., hamster, murine, insect, porcine, bovine or ovine) or tissue origin (e.g., lung, liver, lymphoid, mesenchymal or epidermal) than the MHC source are routinely screened for the ability to introduce variant glycosylation as characterized for example by elevated levels of mannose or variant ratios of mannose, fucose, sialic acid, and other sugars typically found in mammalian glycoproteins. In vitro processing of the subunit typically is accomplished by enzymatic hydrolysis, e.g., neuraminidase digestion.

MHC glycoproteins suitable for use in the present invention have been isolated from a multiplicity of cells using a variety of techniques including solubilization by treatment with papain, by treatment with 3M KCl, and by treatment with detergent. For example, detergent extraction of Class I protein followed by affinity purification can be used. Detergent can then be removed by dialysis or selective binding beads. The molecules can be obtained by isolation from any MHC I bearing cell, for example from an individual suffering from a targeted cancer or viral disease.

Isolation of individual heavy chain from the isolated MHC glycoproteins is easily achieved using standard techniques known to those skilled in the art. For example, the heavy chain can be separated using SDS/PAGE and electroelution of the heavy chain from the gel (see, e.g., Dornmair et al., supra and Hunkapiller, et al., Methods in Enzymol. 91:227-236 (1983). Separate subunits from MHC I molecules are also isolated using SDS/PAGE followed by electroelution as described in Gorga et al. J. Biol. Chem. 262:16087-16094 (1987) and Dornmair et al. Cold Spring Harbor Symp. Quant. Biol. 54:409-416 (1989) Those of skill will recognize that a number of other standard methods of separating molecules can be used, such as ion exchange chromatography, size exclusion chromatography or affinity chromatography.

Alternatively, the amino acid sequences of a number of Class I proteins are known, and the genes have been cloned, therefore, the heavy chain monomers can be expressed using recombinant methods. These techniques allow a number of modifications of the MHC monomers as described above. For instance, recombinant techniques provide methods for carboxy terminal truncation which deletes the hydrophobic transmembrane domain. The carboxy termini can also be arbitrarily chosen to facilitate the conjugation of ligands or labels, for example, by introducing cysteine and/or lysine residues into the molecule. The synthetic gene will typically include restriction sites to aid insertion into expression vectors and manipulation of the gene sequence. The genes encoding the appropriate monomers are then inserted into expression vectors, expressed in an appropriate host, such as E. coli, yeast, insect, or other suitable cells, and the recombinant proteins are obtained.

As the availability of the gene permits ready manipulation of the sequence, a second generation of construction includes chimeric constructs. The α₁, α₂, α₃, domains of the class I heavy chain are linked typically by the α₃ domain of Class I with beta-2 microglobulin and coexpressed to stabilize the complex. The transmembrane and intracellular domains of the Class I gene can optionally also be included.

Construction of expression vectors and recombinant production from the appropriate DNA sequences are performed by methods known in the art. Standard techniques are used for DNA and RNA isolation, amplification, and cloning. Generally enzymatic reactions involving DNA ligase, DNA polymerase, restriction endonucleases, and the like, are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y., 1989. The procedures therein are believed to be well known in the art.

Expression can be in procaryotic or eucaryotic systems. Suitable eucaryotic systems include yeast, plant and insect systems, such as the Drosophila expression vectors under an inducible promoter. Procaryotes most frequently are represented by various strains of E. coli. However, other microbial strains may also be used, such as bacilli, for example Bacillus subtilis, various species of Pseudomonas, or other bacterial strains. In such procaryotic systems, plasmid vectors which contain replication sites and control sequences derived from a species compatible with the host are used. For example, E. coli is typically transformed using derivatives of pBR322, a plasmid derived from an E. coli species by Bolivar et al., Gene (1977) 2:95. Commonly used procaryotic control sequences, which are defined herein to include promoters for transcription initiation, optionally with an operator, along with ribosome binding site sequences, including such commonly used promoters as the β-lactamase (penicillinase) and lactose (lac) promoter systems (Change et al., Nature (1977) 198:1056) and the tryptophan (trp) promoter system (Goeddel et al., Nucleic Acids Res. (1980) 8:4057) and the lambda-derived P_{L} promoter and N-gene ribosome binding site (Shimatake et al., Nature (1981) 292:128). Any available promoter system compatible with procaryotes can be used.

The expression systems useful in the eucaryotic hosts comprise promoters derived from appropriate eucaryotic genes. A class of promoters useful in yeast, for example, include promoters for synthesis of glycolytic enzymes, including those for 3-phosphoglycerate kinase (Hitzeman, et al., J. Biol. Chem. (1980) 255:2073). Other promoters include, for example, those from the enolase gene (Holland, M. J., et al. J. Biol. Chem. (1981) 256:1385) or the Leu2 gene obtained from YEp13 (Broach, J., et al., Gene (1978) 8:121). A Drosophila expression system under an inducible promoter (Invitrogen, San Diego, Calif.) can also be used.

Suitable mammalian promoters include the early and late promoters from SV40 (Fiers, et al., Nature (1978) 273:113) or other viral promoters such as those derived from polyoma, adenovirus II, bovine papilloma virus or avian sarcoma viruses. Suitable viral and mammalian enhancers are cited above.

The expression system is constructed from the foregoing control elements operably linked to the MHC sequences using standard methods, employing standard ligation and restriction techniques which are well understood in the art. Isolated plasmids, DNA sequences, or synthesized oligonucleotides are cleaved, tailored, and religated in the form desired.

Site-specific DNA cleavage is performed by treatment with the suitable restriction enzyme (or enzymes) under conditions which are generally understood in the art, and the particulars of which are specified by the manufacturer of these commercially available restriction enzymes. In general, about 1 μg of plasmid or DNA sequence is cleaved by one unit of enzyme in about 20 μl of buffer solution; an excess of restriction enzyme may be used to insure complete digestion of the DNA substrate. After each incubation, protein is removed by extraction with phenol/chloroform, and may be followed by ether extraction, and the nucleic acid recovered from aqueous fractions by precipitation with ethanol followed by running over a Sephadex G-50 spin column. If desired, size separation of the cleaved fragments may be performed.

Restriction cleaved fragments may be blunt ended by treating with the large fragment of E. coli DNA polymerase I (Klenow) in the presence of the four deoxynucleotide triphosphates (dNTPs) After treatment with Klenow, the mixture is extracted with phenol/chloroform and ethanol precipitated followed by running over a Sephadex G-50 spin column.

Synthetic oligonucleotides are prepared using commercially available automated oligonucleotide synthesizers. In the proteins of the invention, however, a synthetic gene is conveniently employed. The gene design can include restriction sites which permit easy manipulation of the gene to replace coding sequence portions with these encoding analogs.

Correct ligations for plasmid construction can be confirmed by first transforming E. coli strain MM294 obtained from E. coli Genetic Stock Center, CGSC #6135, or other suitable host, with the ligation mixture. Successful transformants can be selected by ampicillin, tetracycline or other antibiotic resistance or by using other markers depending on the mode of plasmid construction, as is understood in the art. Plasmid from the transformants are then prepared, optionally following chloramphenicol amplification. The isolated DNA is analyzed by restriction and/or sequenced by the dideoxy method of Sanger, F., et al., Proc. Natl. Acad. Sci. USA (1977) 74:5463 as further described by Messing, et al., Nucleic Acids Res. (1981) 9:309, or by the method of Maxam, et al., Methods in Enzymology (1980) 65:499.

The constructed vector is then transformed into a suitable host for production of the protein. Depending on the host cell used, transformation is done using standard techniques appropriate to such cells. The calcium treatment employing calcium chloride, as described by Cohen, S. N., Proc. Natl. Acad. Sci. USA (1972) 69:2110, or the RbCl method described in Maniatis, et al., Molecular Cloning: A Laboratory Manual (1982) Cold Spring Harbor Press, p. 254 is used for procaryotes or other cells which contain substantial cell wall barriers. For mammalian cells without such cell walls, the calcium phosphate precipitation method of Graham and van der Eb, Virology (1978) 52:546 or electroporation is preferred. Transformations into yeast are carried out according to the method of Van Solingen, P., et al., J. Bacter. (1977) 130:946 and Hsiao, C. L., et al., Proc. Natl. Acad. Sci. USA (1979) 76:3829.

The transformed cells are then cultured under conditions favoring expression of the MHC sequence and the recombinantly produced protein recovered from the culture.

MHC-binding Peptides

It is believed that the presentation of antigen by the MHC glycoprotein on the surface of antigen-presenting cells (APCs) occurs subsequent to the hydrolysis of antigenic proteins into smaller peptide units. The location of these smaller segments within the antigenic protein can be determined empirically. These MHC-binding peptides are thought to be about 8 to about 10, possibly about 8 to about 11, or about 8 to about 12 residues in length, and contain both the agretope (recognized by the MHC molecule) and the epitope (recognized by T cell receptor on the T cell). The epitope is a linear sequence of about 8 to about 10, possibly about 8 to about 11, or about 8 to about 12 residues in length, that is recognized by the antigen-specific T cell receptor. The agretope is a continuous or noncontiguous sequence that is responsible for binding of the peptide with the MHC glycoproteins. The invention provides systems, kits, and assays for evaluating putative MHC-binding peptides to determine whether such fragments can be incorporated into a ternary complex with an MHC monomer or modified MHC monomer.

Thus, the invention provides screening methods to be used in screening of candidate peptides for use in diagnostic assays, vaccines, and other treatment modalities. Putative MHC-binding peptides for use in the invention methods can be made using any method known in the art, the most convenient being peptide synthesis for fragments of 8 to 12 amino acids in length.

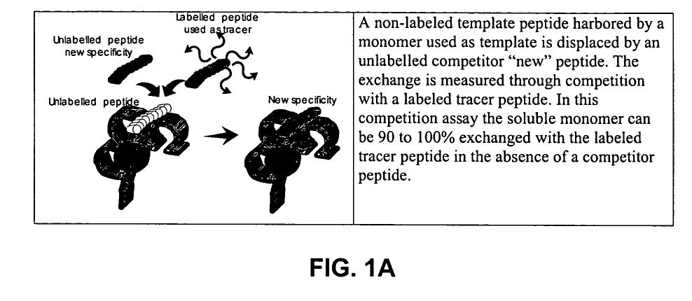

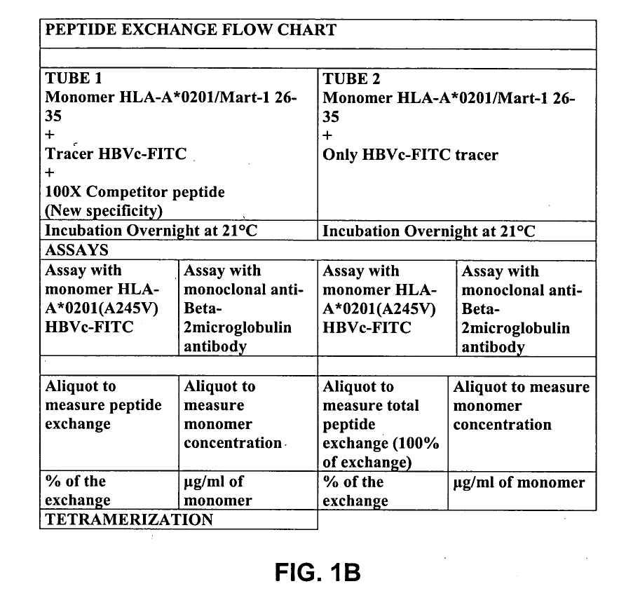

As used herein to describe the invention liquid phase assays, the terms “peptide exchange” and “exchanged peptide” refer to a competition assay wherein three peptides compete in solution for binding to the binding pocket of an MHC monomer or modified MHC monomer. At the start of the competition assay, three peptides are: (1) a MHC-binding peptide, referred to herein as a “template peptide,” which is specific for and is initially bound in the binding pocket of the monomer; (2) an initially unbound test or putative MHC-binding peptide of unknown affinity and/or unknown specificity, referred to herein as a “competitor peptide”; and (3) a detectably labeled, initially unbound “tracer peptide” that is specific for and has a affinity for the binding pocket that is higher than that of the “template peptide”. (See FIG. 1A). The template peptide is selected to have low affinity for the binding pocket so that it is readily is dissociated from the MHC ternary complex and replaced in solution either by a competitor peptide or a tracer peptide. Successful competition of the competitor peptide for the binding pocket indicates the competitor peptide has higher affinity for the binding pocket than either the template peptide or the detectably labeled tracer peptide. Similarly, successful competition of the tracer peptide for the binding pocket indicates the tracer peptide has higher affinity for the binding pocket than both the template peptide and the competitor peptide. Thus, the tracer peptide and template peptide can be selected to establish a minimum affinity for any competitor peptide that is successful in the invention competition assay since a competitor peptide becomes an “exchanged” peptide only if the affinity of the competitor peptide is sufficient under the assay conditions to compete successfully for binding to the binding pocket. A monomer in which the template peptide has been replaced (i.e. exchanged by a higher affinity competitor peptide) is referred to herein for convenience as an “exchanged monomer.” A monomer in which the template peptide has been replaced (i.e. exchanged) by a tracer peptide is referred to herein for convenience as a “tracer monomer.”

In addition to their respective affinities, concentration of the competitor peptide and tracer peptide in the assay solution is also an important consideration in establishing the liquid assay conditions. The competitor peptide is provided in molar excess to allow for optimum binding opportunity, with about 100-fold molar excess being the preferred amount of excess. By contrast, concentration of the tracer peptide is no more than about 0.5 to 1 fold molar excess for example. The concentrations of the tracer peptide and the competitor peptide of the desired specificity are also important. The concentration of the tracer peptide used in the invention assay needs to be low enough to permit peptide exchange of template peptide by the competitor peptide, yet high enough to be detectable under the selected assay conditions if the competitor peptide does not displace the template peptide or displaces only a small portion of the template peptide. The competitor peptide should have a concentration of 100× fold molar excess during the assay to provide for suitable peptide exchange.

The extent to which a competitor peptide replaces the template peptide in the invention competition assay is conveniently assessed by comparing the total amount of signal produced by the label on the tracer peptide in the absence of competitor peptide in the incubation solution with the amount of signal produced by the label on the tracer peptide in the presence of competitor peptide solely by the monomers (i.e., both tracer monomers and exchanged monomers respectively), which can be separated from the solution after the incubation period. The monomers can be washed and separated from the incubation solution using any manner known in the art prior to taking the “monomers alone” signal reading. While the peptide exchange and the first signal detection takes place in solution, the monomers obtained from the incubation solution can be bound to a solid support prior to taking the “monomers alone” signal reading using techniques known in the art and as illustrated in the Examples herein. For example, the flowchart in FIG. 1B illustrates the configuration of the assay, wherein two tubes are used, one without the presence of competitor peptide for establishing the 100% total exchange measure (only HBVc-FITC tracer peptide, which exchanges quasi-totally with the template peptide Mart1 26-35 on the monomer, is in this tube), and the other one containing the competitor peptide to permit measurement of degree of exchange when compared to the 100% tube.

Preferably the monomers are biotinylated, for example as described herein, and bound to avidin-coated wells of microtiter plates or to beads prior to taking the “monomers alone” signal reading. Conventional fluorescence reader can then be used to accurately determine the percentage of the monomers used in the competition assay that became tracer monomers and the percentage that became exchanged monomers. From such determination, the percentage of template peptide that was not displaced or “exchanged” by the competitor peptide and the affinity of the competitor peptide for the monomer binding pocket can be determined mathematically, as exemplified in the Examples herein.

Although the invention solution-based competition assay is illustrated using an HLA-A*0201 monomer as the template monomer, any MHC class I monomer can be selected to serve as the template monomer by observing the following conditions. To select a suitable “tracer peptide” for use in an invention competition it has been determined that the tracer peptide requires sufficient affinity for the monomer pocket in the selected MHC Class I monomer to displace at least 90% of the template peptides in a simple competition assay conducted in solution (and in which a competitor peptide is not present). Thus, those of skill in the art can select a suitable combination of template monomer (including template peptide) and tracer peptide to be used for testing the affinity of any putative MHC-binding peptide of interest, which is used as the competitor peptide in the invention competition assay. In general, the tracer peptide is selected to be of comparatively high affinity for the monomer binding pocket and the template peptide is of comparatively low to medium affinity for the monomer binding pocket.

Depending on the affinity of the competitor peptide, the following scenarios can be encountered.
[A]. Non HLA-A*0201 Peptide Binder.

When the competitor peptide is not an HLA-A*0201 binder, the monomers obtained after peptide exchange should be completely labeled with a fluorescent label.
[B]. Low Affinity HLA-A*0201 Peptide Binder.

When the competitor peptide has low affinity for the monomers, the monomers obtained after peptide exchange are a mixture of monomers bearing the fluorescent peptide and monomers bearing the competitor peptide.
[C]. Medium Affinity HLA-A*0201 Peptide Binder.

When the competitor peptide has medium affinity for the monomers, a low level of fluorescence is detected from the monomers obtained after peptide exchange.
[D]. High Affinity HLA-A*0201 Peptide Binders.

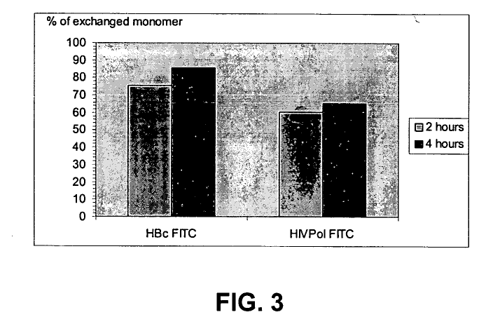

The peptide is considered to have a strong affinity binding capacity for the HLA-A*0201 if the fluorescence is undetectable or poor on the exchanged monomer. These four results are illustrated in FIG. 3.

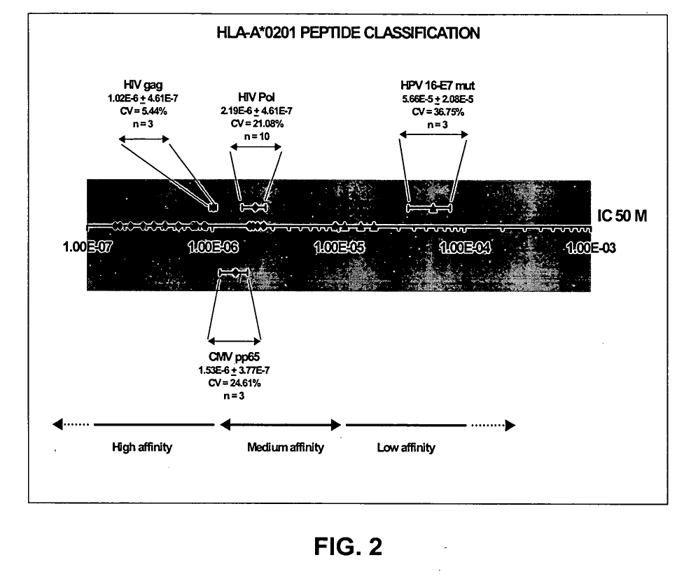

On the basis of the affinity measured with the 4 different peptides (HIVgag, HIVpol, HPV16-E7, CMVpp65 as shown in FIG. 2), the three following classes of peptide affinities have been defined:

high affinity (more than 10⁻⁶M)
medium affinity (between 10⁻⁵ and 10⁻⁶M)
low affinity (Less than 10⁻⁵M)

Accordingly, in one embodiment the invention provides methods for identifying an MHC-binding peptide for an MHC monomer, or modified MHC monomer by incubating under suitable liquid phase conditions a sample comprising: at least one MHC monomer or modified MHC monomer having bound thereto a template MHC-binding peptide, an excess amount of an unbound first competitor peptide, and an unbound tracer MHC-binding peptide tagged with a detectable label. The template peptide is selected to have low or intermediate affinity as compared with that of the tracer peptide for the monomer. Both the tracer peptide and the template peptide are selected for specificity for the monomer used in the assay. In solution, the first competitor peptide, the template peptide, and the tracer peptide compete for binding to the MHC monomer or modified MHC monomer.

The invention peptide exchange assay methods can also be used to determine affinity of the competitor peptide for the monomer by detecting quantitatively and separately the signals produced by the contents of the incubation solution as a whole, and solely by the monomers after they are separated from the incubation solution. The mathematical computation of competitor peptide affinity is illustrated herein in the Examples.

In another embodiment, the invention provides methods for measuring affinity of MHC-binding peptides for an MHC monomer, or modified MHC monomer by incubating under suitable liquid phase conditions a sample comprising: at least one MHC monomer or modified MHC monomer having bound thereto a template MHC-binding peptide, a molar excess amount of a first competitor peptide, and a tracer MHC-binding peptide tagged with a detectable label. The template peptide has lower affinity than the tracer peptide for the monomer. After competition between the first competitor peptide, the template peptide, and the tracer peptide for binding to the MHC monomer or modified MHC monomer at least a portion (or up to the totality) of the first competitor peptide exchanges with the template peptide.

In yet another embodiment, the invention provides methods for measuring function of an MHC-monomer or modified MHC monomer bound to an exchanged peptide for binding to a cell displaying a peptide-restricted T-cell receptor (TCR). This peptide functionality assay is conducted by incubating together under suitable liquid phase conditions a sample comprising: MHC monomers or modified MHC monomers having bound thereto a template MHC-binding peptide, an excess amount of a first competitor peptide, and a tracer MHC-binding peptide tagged with a first detectable label so as to allow competition between the first competitor peptide, the template peptide and the tracer peptide for binding to the MHC monomer or modified MHC monomer, wherein the template peptide has lower affinity than the tracer peptide for the monomer. In at least a portion of the monomers, the first competitor peptide exchanges with the template peptide to form exchanged monomers. A multimer of the exchanged monomers is formed by binding the exchanged monomers with a multivalent entity labeled with a second detectable label; and binding of the exchanged monomers in the multimer with the TCR of the cell is then determined, wherein the binding indicates the first competitor peptide in the exchanged monomers is specific for the TCR. For instance if the TCR is specific for

It is presently preferred that the monomers are biotinylated for ease of formation of a tetramer or other multimer. Multimers of exchanged monomers are preferably tagged with a moiety than can be used to attach the tagged monomers to a multivalent core entity. For example, if the monomers used in the assay can be tagged with biotin, the multimer can then be formed by binding of the monomers to an avidinated multivalent entity, such as a cell surface, a liposome, and the like. Preferably, the multimer is formed by binding of the biotinylated exchanged monomers to streptavidin or avidin to form tetramers, which are detectably labeled with PE. Determination of tetramer “staining” of the TCR-bearing cells is readily then determined using flow cytometry, as is illustrated in the Examples herein.

In still another embodiment, the invention provides systems useful for identifying an MHC-binding peptide for an MHC monomer, or modified MHC monomer. The invention systems include at least one MHC monomer or modified MHC monomer having bound thereto a template MHC-binding peptide, and a tracer MHC-binding peptide tagged with a detectable label, wherein the template peptide has lower affinity than the tracer peptide for the monomer. The invention system may further include an instruction for using the system.

The suitable liquid phase conditions used in the invention peptide exchange assays include, for example, incubating the sample for about 2 to 6 hours or preferably 15 to 20 hours (or overnight) at room temperature (about 21° C.). The pH of the incubation solution is preferably maintained in a range high enough to avoid denaturation of the monomer, for example about pH 8.0.

The invention peptide exchange assay has a number of utilities. Using a third labeled peptide of known affinity in a competition solution-based assay, the exchange reaction can be measured by observing the degree to which the labeled peptide out-competes the test peptide. Thus, the invention is useful in epitope discovery programs commonly known as “epitope screening assays”, which are designed to identify good binding epitopes for a given allele, to correlate affinities with folding yields, and for correlation of affinities with stability of product. In addition, a standard allele monomer can be used in the invention methods to manufacture monomers and tetramers in a rapid and cost-effective way.

The following examples are intended to illustrate, but not limit, the invention.

### EXAMPLE 1

This example illustrates selection of the monomer/peptide combination to be used as template monomer for the peptide exchange assays. Experiments were carried out with HLA A*0201 and a series of known peptides. Previous experiments in the laboratory indicated the peptide Mart-1 27-35 (Kawakami, et al., 1994a Proc. Natl. Acad. Sci. USA. 91: 3515-3519) as well as peptide Mart-1 26-35 (Kawakami, et al., 1994b J. Exp. Med. 180: 347-352), derived from melanoma cells, could be excellent candidates to manufacture monomer template. Both peptides, which have amino acid sequences AAGIGILTV and EAAGIGILTV respectively, have been described as a low and medium affinity peptides for the HLA-A*0201 molecule (Valmori, et al. 1998 (1998) J. Immunol. 161:6956-6962; Kuhns, et al., 1999 J. Biol. Chem. 274(51):36422-36427; Men, et al., 1999 J. Immunol. 162:3566-3573).

The HIVpol peptide (Tsomides, et al., 1991 Proc. NatL. Acad. Sci. USA. 88:11276-11280), which naturally has a lysine in position 3 that was labeled with the FITC molecule, was also tested and it was found that the labeled peptide folds well with the HLA-A*0201 heavy chain. The HBVcore-FITC and the HIVpol-FITC peptides were used to monitor peptide exchange.

### Materials and Methods

Reagents.

Fine chemicals, unless otherwise stated, were obtained from Merck and Carlo Erba. Biotinylated BSA as well as avidin was obtained from Immunoanalysis manufacturing service of Immunotech. LUMITRAC-600® White 96-well microtiter plates were from Greiner ([PN: 655074 LUMITRAC® 600).

Preparation of Avidin Coated 96-well Microtiter Plates.

Each well of white 96-well microtiter plates were coated with 200 μl of a 10 μg/ml biotinylated BSA solution in PBS and the plates were incubated for 16 hours (overnight) at room temperature (20 to 25° C.). The plates were washed and 200 μl/well of avidin solution at 10 μg/ml was added. The plates were incubated 16 hours (overnight) at room temperature (20 to 25° C.). The plates were washed and a blocking, drying solution was added. The plates were incubated 16 hours (overnight) at room temperature (20 to 25° C.). After this time, the solution was poured off and the plates were slapped face down on paper towels. The plates were placed in a special drying room for 24 hours. After this, the plates were placed individually in the self-lock bag until use. The white plates have been designed to have high protein binding capacity [600 ng/cm²]. Thus the avidin-coated plates capture the biotinylated monomer reaching equilibrium in one hour. It is very well know that the Kd of the avidin-biotin reaction is extremely high (˜10⁻¹⁴ M).

In Solution Peptide Exchange Procedure.

Peptide HBc 18-27 (FLPSDC(FITC)FPSV (Phe-Leu-Pro-Ser-Asp-Cys-Phe-Pro-Ser-Val) (Van der Burg, et al., 1995, 1996) was selected as tracer peptide and different concentrations of the competitor peptide (to be tested for desired peptide specificity). In most of the cases when tetramers were manufactured after the exchange, the competitor peptide was added at 100× fold molar excess. The invention peptide exchange reaction was carried out with the monomers and peptides in solution (10 mM Tris, 150 mM NaCl, 0.5 mM EDTA; 0.1% NaN₃, 0.2% BSA; pH 8.0). The mixture was incubated overnight (15 to 20 hours)at 21° C. (controlled temperature) under shaking and protected from light. One aliquot was taken to determine the peptide exchange yield. The rest of the sample was tetramerized with the SA-PE as described below.

Measurement of the Peptide Exchange Yield.

Measurement of peptide exchange yield was conducted as follows: 200 μl/well of standard fluorescent monomer HBVc-FITC and samples containing the monomers from the exchange reaction at 0.25 μg/ml (diluted in Tris 10 mM, NaCl 150 mM, EDTA 0.5 mM, NaN3 0.1%, BSA 0.2%; pH 8.0)) were loaded onto the avidin-coated plate and were incubated for 1 hour at room temperature on an orbital shaker in the dark. Total fluorescence was read and then the monomers were rinsed three times with an automatic washer (SLT, Salzburg, Austria) with 300 μl of a 9 g/l NaCl solution containing 0.05% Tween 80 to remove unbound components. Two hundred μl/well of Tris 10 mM, NaCl 150 mM, EDTA 0.5 mM, NaN3 0.1%, BSA 0.2%; pH 8.0 were added. Bound fluorescence was read with a Perkin Elmer LS-50B fluorometer following these parameters: λ Excitation=495 nm; λ Emission=525 nm; Emission filter=515 nm; Band pass (Exc, Emis)=10, 15 nm; rate: 0.5 sec/well.

Measurement of the Monomer Concentration.

The fluorometric assay procedure was as follows: 200 μl/well of standard monomer HBVc-FITC and samples containing the monomers at 0.25 μg/ml (diluted in Tris 10 mM, NaCl 150 mM, EDTA 0.5 mM, NaN3 0.1%, BSA 0.2%; pH 8.0) were loaded onto the avidin-coated plate and incubated for 1 hour at room temperature on an orbital shaker in the dark. The plates were rinsed three times with an automatic washer (SLT, Salzburg, Austria) with 300 μl of a 9 g/l NaCl solution containing 0.05% Tween 80. Two hundred μl/well of 2 μg/ml of anti-beta-2 microgrobulin B1G6 mAb coupled to the Phycoerythrine diluted in Tris 10 mM, NaCl 150 mM, EDTA 0.5 mM, NaN3 0.1%, BSA 0.2%; pH 8.0 were added and incubated for 2 hours at room temperature on an orbital shaker in the dark. The plates were rinsed 3 times as described above and wells were filled with 200 μl of Tris 10 mM, NaCl 150 mM, EDTA 0.5 mM, NaN3 0.1%, BSA 0.2%; pH 8.0. The PE fluorescence bound to the monomer was measured with a Perkin Elmer LS-50B fluorometer using these parameters: λExcitation=488 nm; λEmission=575 nm; Emission Filter=515 nm; Band pass (Exc,Emis)=5, 8 nm; 0.5 sec/well.

Tetramerization of the Exchanged Monomer.

The exchanged monomers as well as control monomers were tetramerized with SA-PE (ratio of 0.25) (as described in U.S. Pat. No. 5,635,363, which is incorporated herein by reference in its entirety. The exchanged monomers were mixed at 200 μg/ml of monomer in 10 mM Tris, 150 mM NaCl, 0.5 mM EDTA; 0.1% NaN3, 0.2% BSA with the solution of SA-PE. The final concentration of the monomers was 100 μg/ml. After homogenization the sample was incubated 15 min at room temperature. After that the sample was incubated at 4° C. in the dark.

Flow Cytometry Assay.

Staining of control cells was performed according to the following staining procedure. About 5×10⁵ cells/test were stained with 10 μl of tetramers (1 μg/test) by incubating the cells for 30 min at 4-8° C. protected from the light. After cells were washed with 4 ml of 1XPBS, 0.1% NaN3, 0.2% BSA, cells were centrifuged 5 min at 1200 rpm and the supernatant was discarded. Cells were resuspended in 0.5 ml 1XPBS 0.5% FA. Samples were acquired on EPICS XL cytometer (Beckman Coulter, Inc., Fullerton, Calif.).

Selection of Monomer HLA-A*0201(A245V)/Mart-1 27-35 or Mart-1 26-35 as Template and Selection of Tracer Peptide HBVc-FITC or HIV pol-FITC.

To determine whether the monomer HLA-A*0201 (A245V) folded with either the peptide Mart-1 27-35 or Mart-1 26-35, can be used as template monomer and can accept fluorescent tracer molecules, the monomer Mart-1 27-35 was incubated in solution in presence of fluorescent peptides, HBVc-FITC and HIVpol-FITC, both of which fold well with the HLA-A*0201 heavy chain.

HBc 18-27-FITC (FLPSDC(FITC)FPSV) (Van der Burg, et al., 1995 Human Immunol. 44:189-198; and Van der Burg et al. 1996 J. Immunol. 156: 3308), has a tyrosine in position 6 replaced by a cysteine for labelling with the fluorescent dye FITC.

The FITC molecule was selected to label the peptides because FITC is a small molecule (MW 389 daltons) compared to the PE molecule, can be coupled easily to the peptides, and the small size avoids steric hindrances between the FITC molecule and the heavy chain during the folding reaction. Most importantly, FITC molecule has very high yield, compared to other fluorochromes tested, such as Alexa dyes or others.

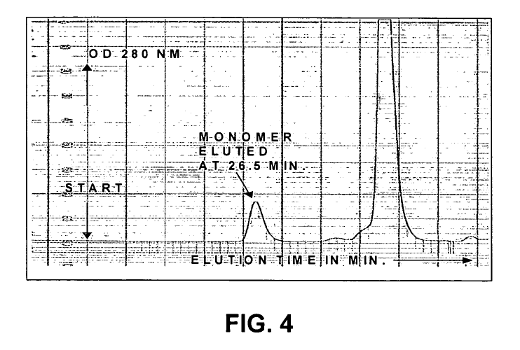

Monomers and fluorescent peptides were mixed and incubated 2 and 4 hours in solution to allow peptide exchange. After this time the monomers were loaded into avidin plates prepared as described above and after 1 hour of incubation the plates were washed and the bound fluorescence detected. Both fluorescent peptides substantially bind to the monomer Mart-1 27-35, as shown in FIG. 4.

It was noted that after 4 hours the HBVc-FITC peptide exchanged at approximately 90% compared to HIVpol-FITC peptide, which exchanged about 70%. These findings allowed testing in more detail of the conditions of the exchange and suggested that this method can be used to measure the peptide exchange yield. The HBVc-FITC peptide was chosen for use as tracer peptide in the competition assays based on its higher exchange rate of about 90%.

Determination of the Integrity of the Exchanged Monomer.

These results showed that monomers remain intact after the peptide exchange. However, to verify this point, the fluorescent-exchanged monomer was loaded into a Superdex® 75 column and the elution volume was fractionated. The absorbance at 280 nm was recorded. Chromatographic profile is shown in FIG. 4.

A peak corresponding to the typical elution time range of the monomer on Superdex 75. (26.5 min). was observed. The major peak corresponds to the excess of peptide. This data demonstrates formally that the monomer remains associated during and after peptide exchange.

Optimization of the Exchange.

Kinetic exchange experiments were carried out using different concentrations of both HBVc-FITC and HIVpol-FITC peptides in the solution peptide exchange. The experiments were carried out with Monomers Mart-1 27-35 and 26-35 as well as with monomers HIVpol and CMVpp65. The HIVpol and CMVpp65 peptides are known to be medium to high affinity peptides. The HIVpol and CMVpp65 monomers were included in the assay to support the use of the monomers Mart-1 27-35 and 26-35 as template monomers in the invention exchange assay.

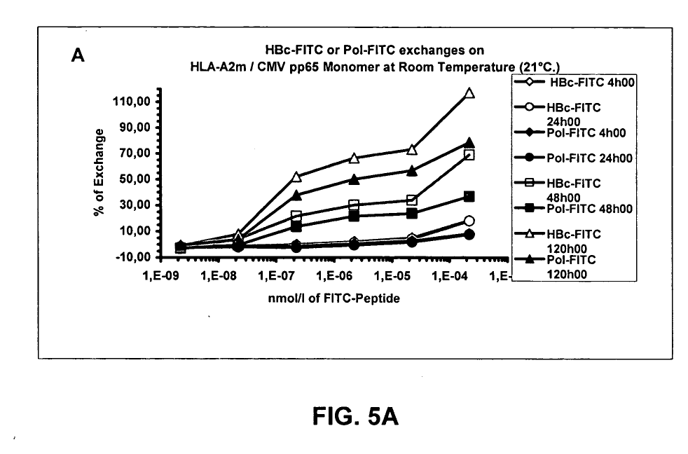

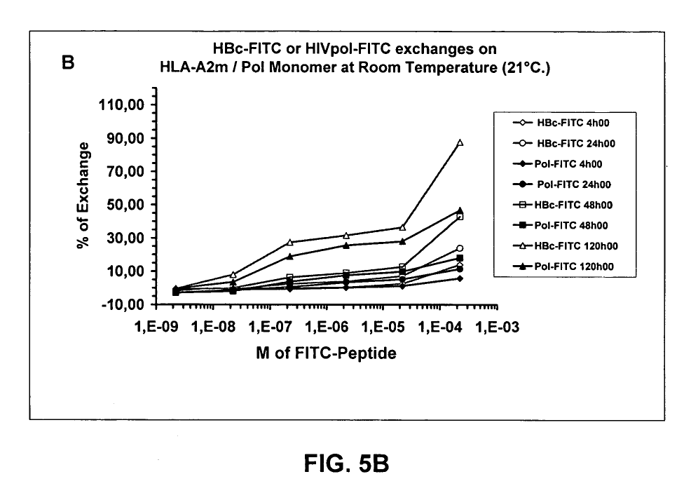

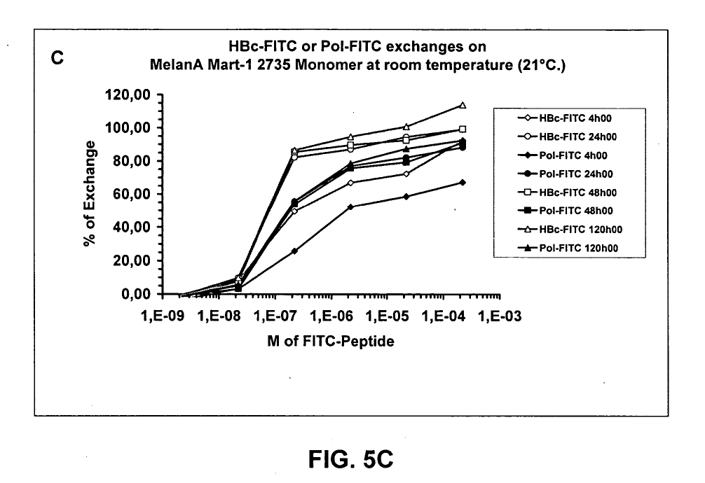

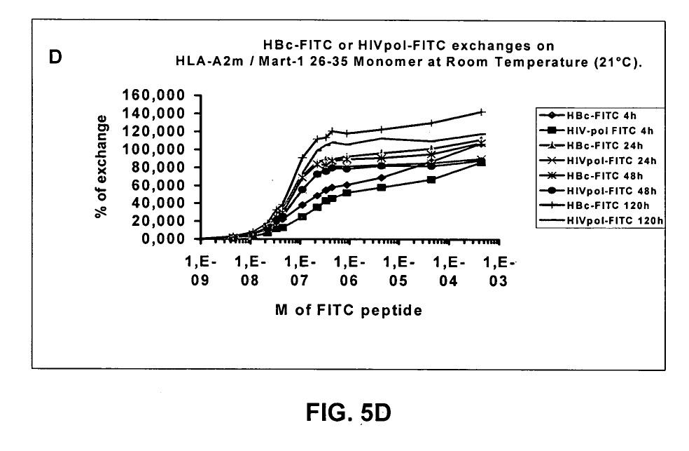

The results of the exchange assay (FIGS. 5A-D) showed that the % exchange was weak when HIVpol and CMVpp65 monomers were used as template (FIGS. 5B, and 5C, respectively). In contrast, the % exchange was higher and faster compared to the other two monomers when monomers Mart-1 26-35 and Mart-1 27-35 were used (FIGS. 5A and 5D, respectively).

After 4 hours, about 90% of the peptide was exchanged when tracer peptide HBVc-FITC was at a concentration of at 200 nM in the exchange medium with both monomers HLA-A*0201 (A245V)/Mart-1 27-35 or Mart-1 26-35. The exchange of the HBVc-FITC tracer peptide on HIVpol and CMVpp65 monomers was totally different than the exchange observed on the Mart-1 monomers. The maximum percentage of exchange obtained with the HBVc-FITC peptide on the monomer HIVpol was 40%. The value obtained with a 200 nM concentration of HBVc-FITC after 120 h of incubation is rather an outlier. The CMVpp65 monomer allowed better exchange (about 80% after 120 h) than the HIV pol monomer. These results confirmed choice of the Monomer Mart-1 27-35 and monomer Mart-1 26-35 to be used as template monomers for peptide exchange assays.

However, based upon prior experience and internal stability data, monomer Mart-1 26-35 was selected for use as the template monomer because the peptide Mart-1 26-35 folds better than the peptide Mart-1 27-35, the final monomer yield after folding and purification is better, and the monomer stability is better when peptide 26-35 was used as the template peptide in the template monomer. It was discovered that the low affinity of peptide Mart-1 27-35 compared to the intermediate affinity of peptide Mart-1 26-35 in fact explains why peptide Mart-1 26-35 folds better.

From these results the fluorescent peptide selected as best to be used as the tracer peptide was the peptide HBVc-FITC because it has high enough binding affinity for the folded HLA-A*0201 heavy chain to permit the exchange with the template peptide (Mart1 26-35) on the monomer as well as being detectable if the competitor peptide doesn't bind to the template monomer.

Quantification of the Level of the Peptide Exchange.

The quantification of the peptide exchange was performed in an avidinated 96-well microtiter plate prepared as described above. HLA-A*0201/HBVc-FITC monomer was used as a standard. Serial two fold dilutions of monomer HLA-A*0201/HBVc-FITC were performed and run at the same time as the exchanged monomer.

In a previous experiment it had been discovered that the monomers HLA-A*021/Mart-1 27-35 and Mart-1 26-35 exchanged more than 85% with 0.5× of HBVc-FITC peptide, because the FITC-signal of the exchanged monomer without competitor peptide produced a signal similar to the signal obtained using the same concentration of the monomer directly folded (not by peptide exchange) with the HBVc-FITC peptide.

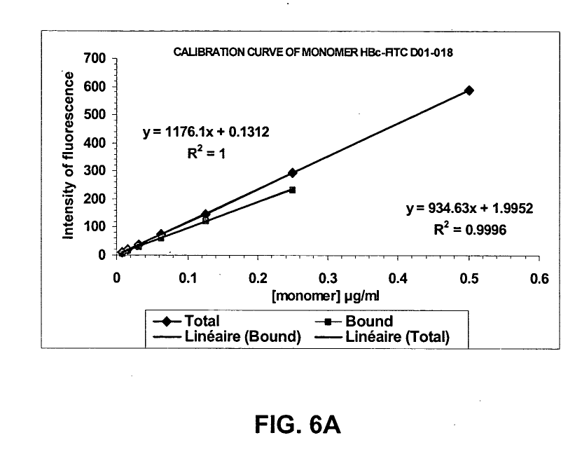

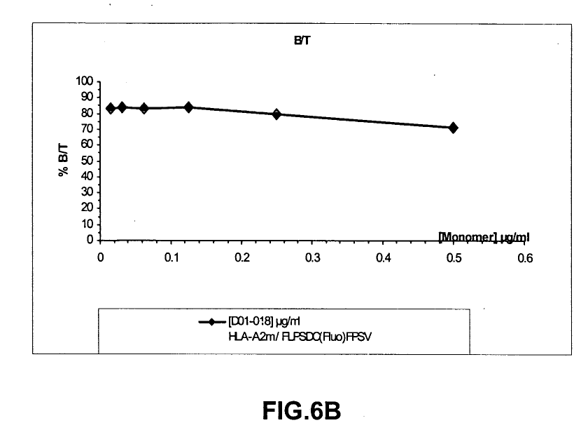

In order to quantify the exchange, two different tubes were run for each test. One tube contained the monomer Mart-1 26-35 with 100-fold molar excess of competitor peptide (peptide of desired specificity) and 0.5-fold molar excess of HBVc-FITC tracer peptide. A second tube was run in parallel without the competitor peptide and containing only the HBVc-FITC tracer peptide and the monomer. This second tube permits an accurate measurement of the exchange because it provides the 100% value of the tracer peptide exchanged knowing the HBVc-FITC tracer peptide exchanges substantially totally with the template peptide (Mart-1 26-35) on the monomer. Thus, the remaining fluorescence on the monomer alone after the exchange permits calculation of the concentration of the exchanged monomer from the HLA-A*0201/HBVc-FITC standard curve, and finally, the yield of exchanged monomer when compared to the 100% value obtained with the tube containing only the tracer peptide. An example of the method used to calculate the exchange is shown in Table 1. The typical standard curve using the monomer HBVc-FITC is shown in FIG. 6A in which diamonds correspond to the total fluorescence and squares correspond to the bound fluorescence.

It is important to underscore that the % B/T remains stable until 0.25 μg/ml of monomer HBVc-FITC, indicating that above this concentration the solid phase begins to be saturated. Under these conditions a linear dose response curve was obtained up to 0.25 μg/ml of the monomer. The detection limit, defined as the minimal concentration of monomer HLA-A*0201/HBVc-FITC that produces a signal equal to the nonspecific background plus 3 standard deviations, was as low as 0.00278 μg/ml (2.78 ng/ml). This value was calculated from the data obtained from 6 different assays carried out with three different lots of plates and carried out on different days.

The different samples were diluted at 0.25 μg/ml of monomer. The results were plotted and the equation of the linear regression corresponding to the bound minus the non specific binding (MFI-NSB) was obtained. From these calculations, the concentration of the monomer in the samples was calculated. The values were corrected by the % of B/T of the value obtained with 0.25 μg/ml from the standard curve. Lastly the % of the exchange was calculated, using the value obtained from the samples without competitor as the 100% value. In this experiment, peptide HIVpol exchanges at 87.21%.

An example of the quantification of the peptide exchange using the HIVpol peptide is shown in the Table 1 below.

TABLE 1
Quantification of the exchange.

| | [monomer] | | | | | | | % Exchanged |
|---|---|---|---|---|---|---|---|---|
| | μg/ml | | | | NSB | Conc. | B/T | Monomer |
| | Loaded | 1 | 2 | Mean | Subtract | μg/ml | Correction | vs No Comp. |
| With 100x | | | | | | | | |
| Competitor | | | | | | | | |
| Bound | 0.25 | 103.3 | 102.9 | 103.1 | 20.25 | 0.020 | 0.0245 | 87.21 |
| | NSB | 82.8 | 82.9 | 82.85 | 0 | | | |
| No Competitor | | | | | | | | |
| Only tracer | | | | | | | | |
| Bound | 0.25 | 226.6 | 228.6 | 227.6 | 144.75 | 0.153 | 0.1916 | |
| | NSB | 82.8 | 82.9 | 82.85 | 0 | | | |

However, it was noted that the concentration of the monomer in the sample incubated only with the tracer peptide was not 0.25 μg/ml as expected, indicating either that a fraction of the monomer was dissociated or that the monomer was not totally exchanged.

### EXAMPLE 2

To understand better what happened during the exchange, the anti-beta-2 microglobulin monoclonal antibody B1G6, which recognizes an epitope located outside the interface of interaction between the heavy chain and beta-2 microglobulin, was coupled to PE and used to quantify the exchanged monomer accurately as well as to have an idea of the amount of monomer dissociation that took place.

Measurement of Monomer Dissociation Level and Quantification of Total Monomer.

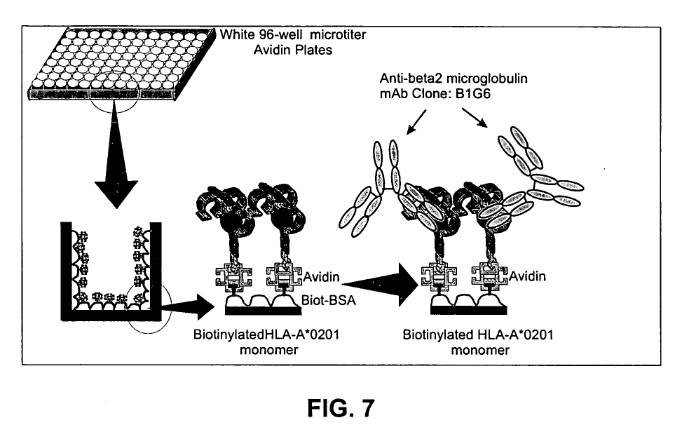

In this immunometric assay, as illustrated in FIG. 7, the monomer is bound first to the avidin coated plates via a biotin tag engineered at the C-terminal end of the heavy chain. After the plates were washed, the B1G6 mAb was added so as to allow binding of the beta-2 microglobulin associated with the heavy chain in the monomer. The immunometric assay was performed in two steps. The first step involves incubation of the monomer for coating. Then, after washing to remove unbound components, and particularly free B2-microglobulin, the second step involves incubation with the B1G6-PE to reveal bound monomer. In this way interference of free beta-2 microglobulin with the antibody, which could cause under estimation of the concentration of the monomer, is avoided.

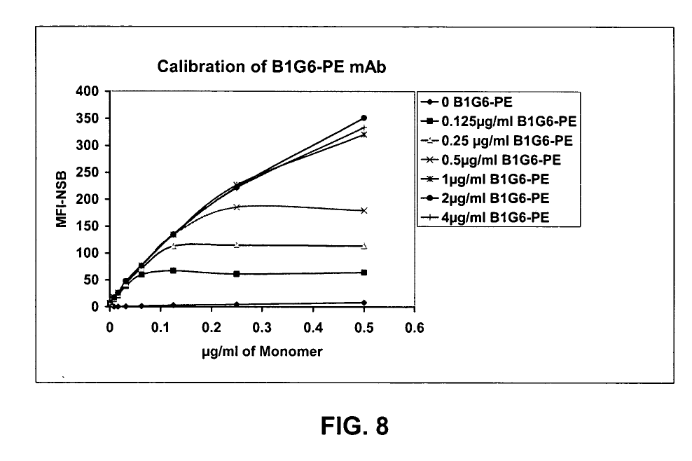

In the immunometric assay the anti-beta-2 microglobulin B1G6 mAb was coupled to PE. Prior to use in this assay several experiments were carried out to validate the antibody for use in the monomer assay as follows. To determine the best concentration of the B1G6-PE and the dynamic range of the standard curve, serial two-fold dilutions of HLA-A*0201/HBVc-FITC monomer as well as serial two-fold dilutions of B1G6-PE were tested using the protocol as described in the material and methods section. Results are shown in FIG. 8. The dose-response curve obtained reached a plateau at 1 μg/ml with the plates coated with the monomer HLA-A*0201/HBVc-FITC and a saturation of the signal when the anti-beta-2m mAb was added at less than 1 μg/ml. It was noted that at 1, 2 and 4 μg/ml of anti-beta-2m mAb the signal increased compared to the signal obtained at 0.5 μg/ml of the Ab. The bivalent nature of the antibody easily explains this phenomenon. The antibody B1G6 was lower than the concentration of the monomer, the two binding sites of the antibody binding two beta-2 ms.

However, when the antibody was in excess, only one binding site of the antibody binds to one beta-2m, increasing the number of antibodies attached to the plate and, in consequence, increasing the signal. This result was observed when signals obtained with 0.5 μg/ml and 1 μg/ml of anti-beta-2m mAb and 0.5 μg/ml of monomer (180 MFI versus 321 MFI, respectively) were compared.

Taken together, these data suggest using 1 to 2 μg/ml of B1G6-PE mAb to test for monomer dissociation (the dynamic range of the standard curve being 0.25 μg/ml to 0 μg/ml of monomer).

It is important to underscore that the PE signal was not contaminated by the FITC signal, despite both fluorochromes being excited at 488 nm, because the FITC emission was recorded at 525 nm and the PE signal emission was recorded at 575 nm. This aspect was verified by exciting the HBVc-FITC monomer and recording the emission at 575 nm. Results showed that no signal or only signal similar to the background was obtained under these conditions.

Another point that was necessary to consider in designing this assay is the correlation of the monomer concentration measured by OD at 280 nm and the monomer concentration obtained with the B1G6 assay because the two assays are a little bit different. The B1G6 assay depends on the dissociation or the integrity of the monomer and the stability of the epitope recognized by the antibody. In contrast, when the concentration of the monomer is measured by OD, it is impossible to know the state of the monomer because the OD is the same whether the monomer is dissociated or not.

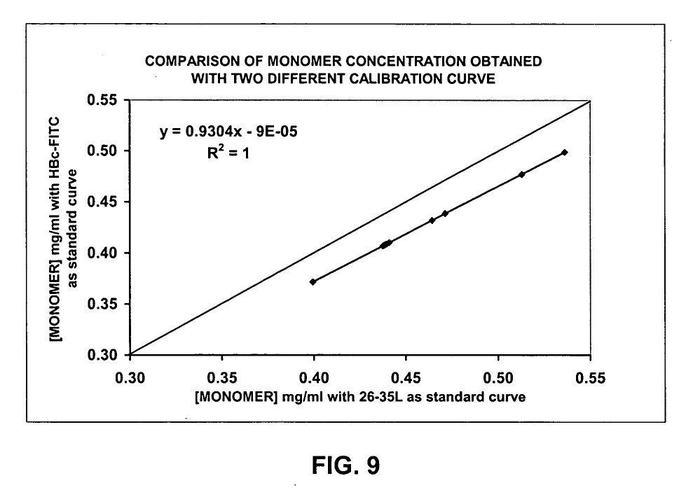

To verify this point, the concentration of different monomers was determined using the invention assay and by OD at 280 nm. In addition, the monomers were quantified using two different monomers, the monomer HLA-A*0201/HBVc-FITC and the monomer HLA-A*0201/Mart-1 26-35L, with which standard calibration curves were realized. Results are shown in Table 2 below, which shows that the CV measured by two methods (i.e., on avidin plates with coated monomers revealed with the Anti-B1G6-PE and by OD at 280 nm) is more homogenous and less important in samples tested with the monomer 26-35L than the values obtained with the monomer HBVc-FITC when each is compared with the OD at 280 nm. Comparison of the values of concentration obtained with these two monomers (FIG. 9) shows a high correlation (R²=1), but does not approach the optimal correlation (shown by the diagonal on the plot). Use of the linear equation suggests that all the monomers are equally recognized by the antibody, but when 1 mg of any monomer was calculated using the monomer 26-35L as standard, there was a differences of (1−0.93=0.07 mg) calculated when using the HBVc-FITC monomer as standard. In other words, there is a 7% error in the determination when the HBVc-FITC monomer is used as the standard.

TABLE 2
Quantification of the concentration of different
monomers by ELISA and OD.

| | | | | B1G6-PE | |
|---|---|---|---|---|---|
| | | | B1G6-PE | [Mono] | |
| | | | [Mono] | final | |
| | | | final | mg/ml | OD |
| | | | mg/ml | Monomer | 280 nm |
| | | | Monomer2 | HBVc- | [Mono] |
| Number | Lot | Specificity | 635 L | FITC | μg/ml |
| 1 | M00-002 | 26-35 L | 0.513 | 0.477 | 0.507 |
| 2 | M00-005 | HIV pol | 0.472 | 0.439 | 0.467 |
| 3 | M00-044 | 26-35 L | 0.400 | 0.372 | 0.502 |
| 4 | M00-074 | CMV pp65 | 0.439 | 0.408 | 0.478 |
| 5 | M00-100 | HIV gag | 0.464 | 0.432 | 0.493 |
| 6 | M00-101 | HIV gag | 0.440 | 0.409 | 0.490 |
| 7 | M00-109 | HIV | 0.438 | 0.407 | 0.475 |
| 8 | M00-110 | MelanA | 0.441 | 0.410 | 0.470 |
| 9 | D01-018 | HBVc-FITC | 0.536 | 0.499 | 0.499 |

| B1G6 Monomer HBVc-FITC/OD | | | B1G6 Monomer 26-35 L/OD | | |
|---|---|---|---|---|---|
| Mean | SD | CV | Mean | SD | CV |
| 0.492 | 0.021 | 4.26 | 0.510 | 0.004 | 0.86 |
| 0.453 | 0.020 | 4.40 | 0.469 | 0.003 | 0.72 |
| 0.437 | 0.093 | 21.18 | 0.451 | 0.073 | 16.15 |
| 0.443 | 0.050 | 11.26 | 0.458 | 0.028 | 6.16 |
| 0.462 | 0.043 | 9.29 | 0.479 | 0.020 | 4.19 |
| 0.450 | 0.058 | 12.80 | 0.465 | 0.036 | 7.71 |
| 0.441 | 0.048 | 10.86 | 0.456 | 0.026 | 5.75 |
| 0.440 | 0.043 | 9.66 | 0.456 | 0.021 | 4.55 |
| 0.499 | 0.000 | 0.02 | 0.517 | 0.027 | 5.14 |

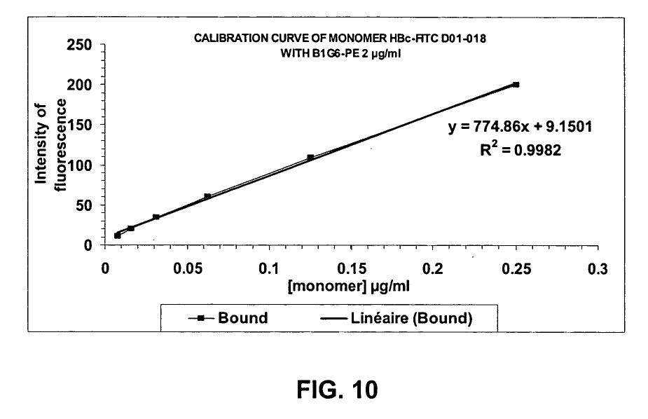

Knowing the limitation of the assay, the monomer concentration of two samples was recalculated as shown in Table 3. The typical standard curve is shown in FIG. 10.

TABLE 3
Concentration of exchanged monomer.

| | [mono- | | | | | | % of |
|---|---|---|---|---|---|---|---|
| | mer] | | | | | | detected |
| | μg/ml | | | | NSB | Conc. | mono- |
| | Loaded | 1 | 2 | Mean | subtract | μg/ml | mer |
| With 100x | | | | | | | |
| Competitor | | | | | | | |
| Bound | 0.25 | 365.5 | 363.7 | 364.6 | 347.35 | 0.188 | 75.2 |
| | NSB | 17.2 | 17.3 | 17.25 | 0 | | |
| No | | | | | | | |
| Competitor | | | | | | | |
| Only tracer | | | | | | | |
| Bound | 0.25 | 353.4 | 354.2 | 353.8 | 336.55 | 0.182 | 72.8 |
| | NSB | 17.2 | 17.3 | 17.25 | 0 | | |

A similar monomer concentration was found in both samples, but not 0.25 μg/ml as expected. When concentrations obtained with the HBVc-FITC monomer and the B1G6-PE assay were compared, close values were obtained (as summarized in Table 4 below). (Mean: 0.189±0.007, % CV=3.73, n=3). These results suggests that a fraction of the monomer dissociated during the incubation.

TABLE 4
Monomer concentration found with HBVc-FITC monomer
and B1G6-PE mAb assays.

| | Assay with monomer | Assay with |
|---|---|---|
| | HBVc-FITC | B1G6-PE |
| 100X competitor and 0.5X | | 0.188 |
| tracer | | |
| No competitor only tracer | 0.196 | 0.182 |

Using this approach the % of the exchange was calculated for the peptide exchange experiments.

### EXAMPLE 3

Peptide Exchange and Cellular Staining.

One of the uses of the invention peptide exchange methods is in the generation of tetramers with a new specificity without doing the entire monomer folding process. To confirm utility of MHC tetramer assays utilizing exchanged monomers for staining T cells, the staining pattern obtained with monomers containing directly folded peptides rather than exchanged peptides was compared. To make that comparison three different peptides for which specific cell lines are available were selected.

Description of Cells Used in this Study.

Mart 1 Specific Cell Line.

Jurkat P1/1 CD8 clone 5.2 Jurkat P1/1 CD8 clone 5.2 is CD3+, CD4+, CD8+, Vb6.7+. The TCR recognizes the Melan A “wild type” peptide (AAGIGILTV) (Mart-1 27-35), but the decamer (EAAGIGILTV) (Mart-1 26-35) and the mutated peptide (26-35L, also called 27L in the literature (ELAGIGILTV) are better recognized. The same type of cell was prepared without CD8. The CD8⁻ cell (called Jurkat P1/1) recognizes the Melan A “wild type” peptide (AAGIGILTV) with extremely low affinity, and is hardly detectable by flow cytometry. Jurkat 1.1 cells clone 5.2, which is specific for the Mart-1 peptides, 27-35, 26-35 and 26-35L restricted to HLA-A*0201 were selected because this cell line can be used to quantify the level of functionally of peptide exchange.

RBL HIVpol: Clone 80210 (Molecular Immunology Group, John Radcliff Hospital, Oxford, England) is a rat basophile leukemia line (RBL), transfected with two hybrid constructs of human TCR alpha and beta chains respectively, fused to the mouse TCR zeta chain (the method was originally described by Engel et al. (1992) Science 256:1318). This configuration allows expression of TCR without need for the CD3 complex, and signalling can be measured directly after TCR engagement. Zeta chains form dimers expressed at the cell surface. The line expresses alpha-alpha and beta-beta homodimers in addition to alpha-beta hetrodimers. There is no CD3 nor CD8 expression on this cell line. The line is adherent, and degranulates upon stimulation (for cytometry: a scatter change upon incubation with anti-TCR reagents is observed). RBL 80210 cells express a specific TCR recognizing the HLA-A*0201/HIVpol peptide complex.

Hybridoma cell CMVpp65 (named N9V2.3) Laboratoire Immunité Cellulaire Antivirale, Institut Pasteur, Paris, France) is a mouse hybridoma recognizing the immunodominant peptide (N9V) of the Cytomegalovirus major tegument protein pp65. A T cell line, 100% positive for the A2/pp65 tetramer, was obtained in transgenic HHD mice after immunization with the N9V peptide in Freund's adjuvant. This line was fused to BW5147 T cell hybridoma. The mice used to obtain reactive T cell express the HLA A2 as a monochain in which alpha 1 and alpha 2 domains are of human A2 molecule, the alpha 3 domain and the membrane and cytoplasmic parts are mouse H2D b, linked to human beta-2 microglobulin (beta-2m). These mice have the mouse H2 D-b locus as well as the mouse beta 2m locus knocked out (Pascaolo S., et al. (1997) J Exp Med 185 (12):2043). T cell hybridoma N9V expresses a specific TCR recognizing the complex HLA-A*0201/CMVpp65.

Hybridoma cell BW-HIVgag (HIVgag) (Laboratoire Immunité Cellulaire Antivirale, Institut PasteurParis, France) is a mouse hybridoma recognizing the immunodominant peptide of the HIV Gag. A T cell line, 100% positive for the A2/HIVgag tetramer, was obtained in transgenic HHD mice after immunization with the HIVgag peptide in Freund's adjuvant. This line was fused to BW5147 T cell hybridoma. The mice used to obtain reactive T cell express the HLA A2 as a monochain in which alpha 1 and alpha 2 domains are of human A2 molecule, the alpha 3 domain and the membrane and cytoplasmic parts are mouse H2D b, linked to human beta-2m. These mice have the mouse H2 D-b locus as well as the mouse beta-2m locus knocked out (Pascaolo S., et al., supra). T cell hybridoma Gag expresses a specific TCR recognizing the complex HLA-A*0201/HIVgag.

These cells were used to determine whether the monomer containing the exchanged peptide stains properly when loaded into an MHC class I tetramer and similarly to that of a tetramer containing a control monomer folded directly with the same specific peptide. In addition, it was anticipated that the staining of Jurkat 1.1 cells clone 5.2 with the exchanged tetramer should give an indication of the level of the exchange.

Concentration of Desired Peptide Specificity.

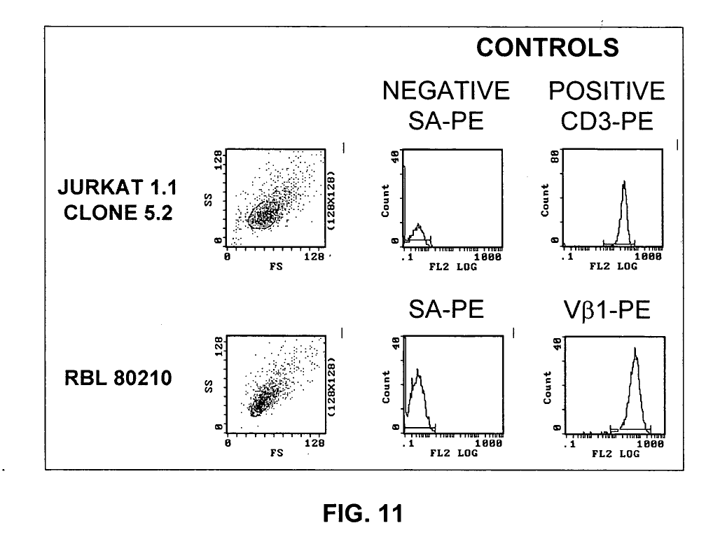

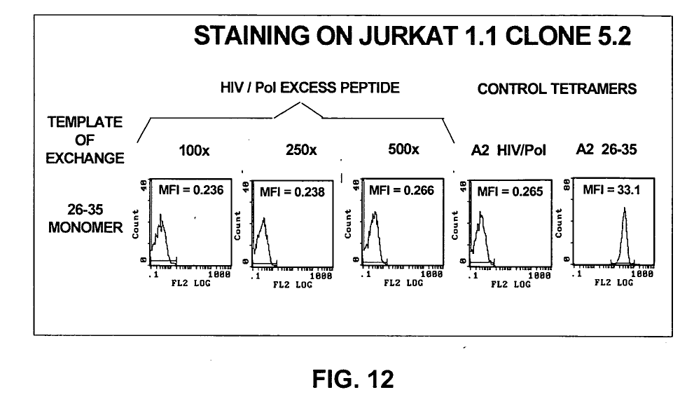

Our previous results showed that 100× fold molar excess is enough to reach a total exchange. To be confident of this result, the HIVpol peptide was used as a model. Three different molar excess concentrations of HIVpol peptide 100×, 250× and 500× were exchanged on monomer 26-35 in presence of 0.5× fold molar excess of HBVc-FITC as tracer. After incubation, the level of the exchange as well as the concentration of the monomer was measured as described above. The exchanged monomer was tetramerized and tested on Jurkat 1.1 cells clone 5.2 and RBL 80210 HIVpol cell line. Results are depicted in FIGS. 11, 12, and 13.

Similar staining was observed independently of the concentration of the HIVpol peptide, suggesting that 100× fold molar excess of peptide is enough to exchange the Mart-1 26 35 peptide on monomer and compete properly with the HBVc-FITC peptide. The signals (MFI) were very close to the signal obtained with the control tetramer, in addition 100% of cells were stained with the tetramers containing exchanged peptides. No staining was found when Jurkat cells were incubated with tetramers containing exchanged peptides, showing that all the original Mart-1 26-35 peptide on the monomer was replaced by the HIVpol peptide.

TABLE 5
Percentage of the exchange.

| | | | | | | | | % Exchanged |
|---|---|---|---|---|---|---|---|---|
| | [monomer] | | | | NSB | Conc. | B/T | Monomer |
| | μg/ml | 1 | 2 | Mean | subtract | μg/ml | correction | vs No Comp. |
| With 500x | | | | | | | | |
| Competitor | | | | | | | | |
| Bound | 0.25 | 100.7 | 100.8 | 100.75 | 16.1 | 0.015 | 0.0182 | 89.88 |
| | NSB | 84.5 | 84.8 | 84.65 | 0 | | | |
| With 250X | | | | | | | | |
| Competitor | | | | | | | | |
| Bound | 0.25 | 95.4 | 95.7 | 95.55 | 10.9 | 0.010 | 0.0121 | 93.28 |
| | NSB | 84.5 | 84.8 | 84.65 | 0 | | | |
| With 100X | | | | | | | | |
| Competitor | | | | | | | | |
| Bound | 0.25 | 104.2 | 103.9 | 104.05 | 19.4 | 0.018 | 0.0220 | 87.73 |
| | NSB | 84.5 | 84.8 | 84.65 | 0 | | | |
| No Competitor | | | | | | | | |
| Bound | 0.25 | 236.2 | 240.3 | 238.25 | 153.6 | 0.149 | 0.1796 | 0.00 |
| | NSB | 84.5 | 84.8 | 84.65 | 0 | | | |

As shown by these results, the % exchange was more than 85%, and the concentration of the total monomer calculated based on use of the B1G6-PE mAb was very close to the concentration calculated based on use of the HBVc-FITC monomer standard curve (0.18 versus 0.179, respectively). The mean±SD of the three exchanges was 90.30±2.80 with a % of CV of 3.10. The concentration of the monomer calculated based on the B1G6 assay was similar in all samples: the % of CV was 3.15 (Table 6).

TABLE 6
Concentration of the monomer measured with the B1G6 assay.

| | μg/ml of |
|---|---|
| Sample | Monomer |
| 500X excess | 0.179 |
| 250X excess | 0.183 |
| 100X excess | 0.183 |
| No competitor | 0.192 |
| Mean | 0.1840 |
| SD | 0.0058 |
| CV | 3.15 |

Contribution of the HBVc-FITC Peptide to the Exchange.

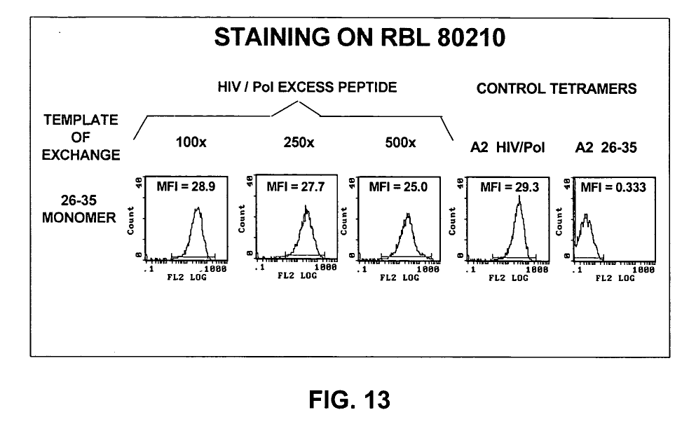

To better understand the contribution of the HBVc-FITC peptide to stabilization of the monomer and the role this peptide played during the exchange, monomer Mart-1 26-35 was incubated with different concentrations of competitor peptide HIVpol and either with or without tracer peptide. After incubation the monomers were tetramerized and tested for staining of Jurkat 1.1 cells clone 5.2 and RBL 80210 cells. The exchange was quantified only for the tubes containing the tracer. The total monomer was calculated based on the B1G6 assay for both types of samples. Flow cytometry results are shown in FIGS. 13A and 13B.

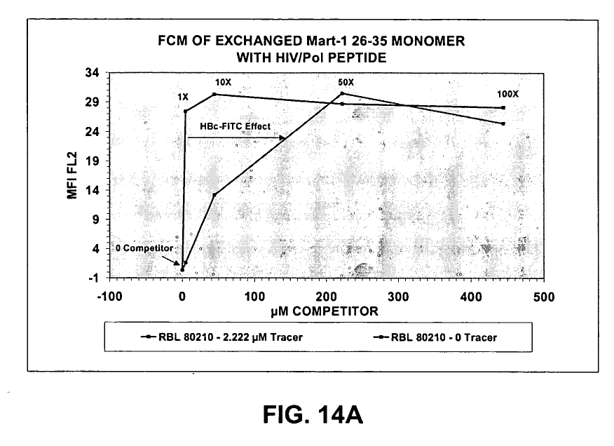

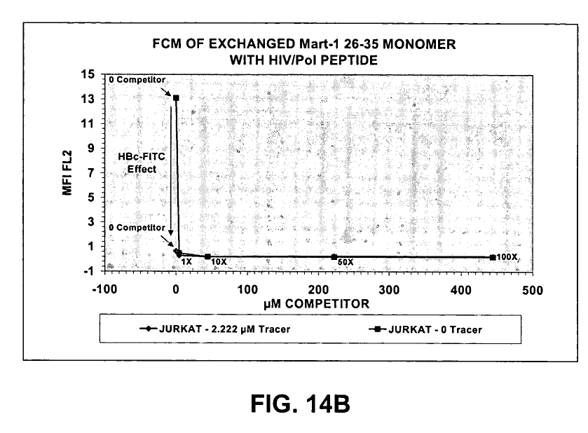

The tetramer made with the exchanged monomer using 133 fold molar excess of competitor peptide in absence of tracer, stained RBL 80210 cells very well and did not stain the Jurkat cells, indicating that the majority of the endogenous peptide Mart-1 26-35 was exchanged. Indeed, the monomer concentration measured with the B1G6-PE test, was similar to that obtained in presence of the tracer. By contrast the tetramer made from monomers exchanged with 1× fold molar excess of HIVpol peptide and in presence of the HBVc-FITC peptide stained only 6.57% of the control, indicating that only a fraction of the total monomer presented the desired specificity. Taken together, these results demonstrated formally that the endogenous peptide can be replaced in an peptide exchange assay and that a real competition takes place. The % of stained cells increased when the competitor concentration was increased. It is interesting to underscore that when the staining was performed with the tetramer exchanged with 50× fold molar excess of competitor peptide and tracer, a result similar to that obtained with the control tetramer was obtained. This data confirms our results shown in FIG. 14, suggesting that template peptides having a low or intermediate affinity compared to the HIVpol peptide can be used in peptide exchange.

The comparison of the data obtained with the biochemical tests showed that the monomer alone (without tracer or competitor) rapidly dissociates, suggesting that both tracer peptides and competitor peptides stabilize the monomer.

TABLE 7
Quantification of the exchange.

| | | | | | | | | % Exchanged |
|---|---|---|---|---|---|---|---|---|
| | [monomer] | | | | NSB | Conc. | B/T | Monomer |
| | μg/ml | 1 | 2 | Mean | subtract | μg/ml | correction | vs No Comp. |
| With 100x | | | | | | | | |
| Competitor | | | | | | | | |
| Bound | 0.25 | 103.3 | 102.9 | 103.1 | 20.25 | 0.020 | 0.0245 | 87.21 |
| | NSB | 82.8 | 82.9 | 82.85 | 0 | | | |
| With 50X | | | | | | | | |
| Competitor | | | | | | | | |
| Bound | 0.25 | 117.4 | 118.3 | 117.85 | 35 | 0.035 | 0.0443 | 76.88 |
| | NSB | 82.8 | 82.9 | 82.85 | 0 | | | |
| With 10X | | | | | | | | |
| Competitor | | | | | | | | |
| Bound | 0.25 | 153.9 | 155.3 | 154.6 | 71.75 | 0.075 | 0.0936 | 51.14 |
| | NSB | 82.8 | 82.9 | 82.85 | 0 | | | |
| With 1X | | | | | | | | |
| Competitor | | | | | | | | |
| Bound | 0.25 | 212 | 216.8 | 214.4 | 131.55 | 0.139 | 0.1739 | 9.25 |
| | NSB | 82.8 | 82.9 | 82.85 | 0 | | | |
| No Competitor | | | | | | | | |
| Bound | 0.25 | 226.6 | 228.6 | 227.6 | 144.75 | 0.153 | 0.1916 | 0 |
| | NSB | 82.8 | 82.9 | 82.85 | 0 | | | |

TABLE 8
Quantification of the monomer with B1G6-PE assay.

| Peptide | | μg/ml | | |
|---|---|---|---|---|
| Molar excess | Tracer | monomer | Tracer | μg/ml monomer |
| 100X | No | 0.157 | Yes | 0.188 |
| 50X | No | 0.174 | Yes | 0.206 |
| 10X | No | 0.163 | Yes | 0.174 |
| 1X | No | 0.157 | Yes | 0.184 |
| 0 | No | 0.059 | Yes | 0.182 |

TABLE 9
Summary of monomer concentration in presence or
absence of tracer.

| | Without | With |
|---|---|---|
| | Tracer | tracer |
| Mean | 0.16275 | 0.1868 |
| SD | 0.008 | 0.012 |
| CV | 4.925 | 6.361 |
| N | 4 | 5 |

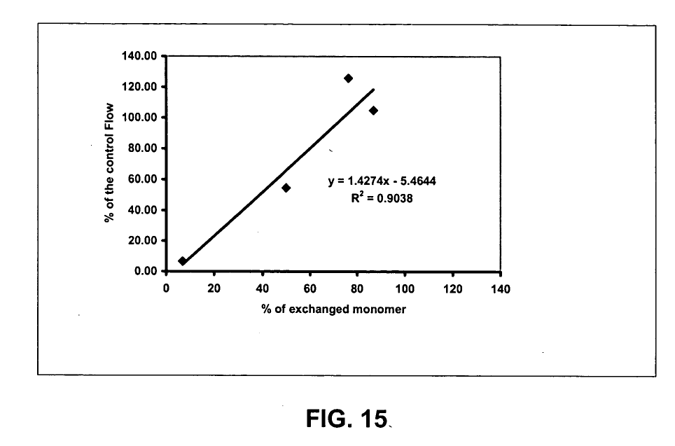

Interestingly a good correlation was found when plotted the % of the control of flow data and the % of the exchanged monomer. (FIG. 15).

Exchange with Peptides from Other Specificities.

The peptide exchange experiments as described above were carried out using peptides HIV/pol, CMVpp65, HIVgag and EBVbmlf1. After incubation the percentage of the peptide exchanged and the concentration of the monomer were measured. The control and exchanged monomers were tetramerized with the SA-PE and used to stain cells.

A high TcR expression on Jurkat 1.1 Clone 5.2 and RBL 80210 cells was observed compared to the TcR expression of N9V (CMV) and Gag Hybridomas. The control tetramers stained specifically their respective cell lines. The SA-PE alone was added as negative control.

No staining was found when Jurkat 1.1 cells clone 5.2 was stained with any of the exchanged tetramers, indicating a total exchange. In each case, the population specifically stained by the exchanged tetramer correlated with peptide specificity of the monomer. However, though the exchanged tetramers stained properly and specifically their respective cells (100% of cells) a slight diminishment on the MFI was observed. See Tables 10 below

TABLE 10
Summary of MFI obtained with different tetramers and different cell lines.

| | Exchanged | Control | Control |
|---|---|---|---|
| | tetramer | tetramer | tetramer |
| | HLA-A*0201/ | HLA-A*0201/ | HLA-A*0201/ |
| Cells | HIVgag | HIVgag | 26-35 |
| Jurkat 1.1 cells Clone | 0.315 | 0.316 | 40.9 |
| 5.2 | | | |
| BW HIVgag | 3.93 | 4.96 (Δ = 0.79) | 0.327 |
| | Exchanged | | |
| | tetramer | tetramer | Control |
| | HLA-A*0201/ | HLA-A*0201/ | tetramer |
| Cells | HIVpol | HIVpol | A*0201/2635 |
| Jurkat 1.1 cells Clone | 0.229 | 0.349 | 40.9 |
| 5.2 | | | |
| RBL 80210 HIVpol | 41.1 | 44.9 (Δ = 0.91) | 0.265 |
| | Exchanged | Control | Control |
| | tetramer | tetramer | tetramer |
| | HLA-A*0201/ | HLA-A*0201/ | HLA-A*0201/ |
| Cells | CMVpp65 | CMVpp65 | 2635 |
| Jurkat 1.1 cells Clone | 0.328 | 0.317 | 40.9 |
| 5.2 | | | |
| N9V CMV pp65 | 3.23 | 4.69 (Δ = 0.68) | 0.262 |

(Δ = MFI Exchanged tetramer/MFI control tetramer)

The major variation was found with tetramers HIVgag and CMVpp65. This variation can be explained by the fact that these cells express at the cell surface, compared to the Jurkat and the RBL 80210 cells, resulting in a low level of the TCR (the signal was obtained in the second decade), and slight variations in the concentration of the monomer might have an influence on the final MFI obtained. The variation was insignificant in the case of HIV pol. This indicates that the dissociation of the monomer should be less dramatic when cells express a high level of TCR, as should be the case of the CD8+T cell lymphocytes.

Quantification of the exchange showed that all monomers exchanged more than 85% (Table 10). The measurement of the total concentration of the monomer indicated that a significant fraction of the monomer dissociates (Table 12). The addition of 10 μg/ml of free beta-2 microglobulin did not improve the level of the exchange and the final concentration of the monomer.

These results can explain, in part, the differences observed by flow cytometry. However, a comparison of concentration of the monomers, calculated with the HBVc-FITC for the samples containing only the tracer peptide (n=4) and those calculated with the B1G6 mAb for all samples (n=8), found a difference of 17% between the values. See Table 13.

These experiments illustrate that peptides exchanged well and stained 100% of cells. Nevertheless, to best understand the effect of the dissociation, an experiment was performed in which the dissociation of the monomer was simulated. In this example, when a fraction of the monomer is dissociated, the dissociated fraction will be unable to stain cells but the dissociated fractions will bind to the streptavidin-PE in the tetramer. Thus, different concentrations of the CMVpp65 folded monomer were tetramerized considering: 0, 10, 20, 30 and 40% of monomer dissociation. The concentration of SA-PE remained constant. Results are shown in Table 14 wherein results are expressed as % of the control. It was observed that the signal remains stable on this tetramer when a fraction of 10% of the monomer is not available. However, when the monomer dissociated at 20% level, 20% of the total signal was lost and so on. These data suggest that a recalculation of the monomer concentration will be necessary before tetramerization to have the same staining profile as is obtained with comparable direct folded monomers.

The compilation of all exchange experiments carried out with at least 100× fold molar excess of competitor peptides shows that peptides exchanged at 89.45%±3.07 with a % of CV of 3.44 for n=9. These statistical results indicate that the peptide exchange protocol is reproducible and accurate.

TABLE 11
Determination of peptide exchange.

| | | | | | | | |
|---|---|---|---|---|---|---|---|
| | | | | | | | % of |
| [monomer] | | | | NSB | Conc. | B/T | exchanged |
| μg/ml | Bound 1 | Bound 2 | Mean | subtract | mg/ml | correction | Monomer |

With HIVpol competitor peptide

| | | | | | | | | |
|---|---|---|---|---|---|---|---|---|
| With | | | | | | | | |
| Competitor | | | | | | | | |
| 100X | | | | | | | | |
| −B2M | 0.25 | 84.2 | 85.2 | 84.7 | 4.95 | 0.005 | 0.0074 | 95.83 |
| +B2m | 0.25 | 84.7 | 85.4 | 85.05 | 5.3 | 0.005 | 0.0081 | 95.69 |
| | NSB | 79.7 | 79.8 | 79.75 | 0 | | | |
| No | | | | | | | | |
| Competitor | | | | | | | | |
| −B2M | 0.25 | 172.5 | 176.4 | 174.45 | 94.7 | 0.1129 | 0.1783 | |
| +B2m | 0.25 | 178.7 | 180.2 | 179.45 | 99.7 | 0.1189 | 0.1878 | |
| | NSB | 79.7 | 79.8 | 79.75 | 0 | | | |

With CMVpp65 competitor peptide

| | | | | | | | | |
|---|---|---|---|---|---|---|---|---|
| With | | | | | | | | |
| Competitor | | | | | | | | |
| 100X | | | | | | | | |
| −B2M | 0.25 | 86.9 | 86.1 | 86.5 | 6.75 | 0.007 | 0.0109 | 94.05 |
| +B2m | 0.25 | 86.6 | 86.4 | 86.5 | 6.75 | 0.007 | 0.0109 | 94.21 |
| | NSB | 79.7 | 79.8 | 79.75 | 0 | | | |
| No | | | | | | | | |
| Competitor | | | | | | | | |
| −B2M | 0.25 | 177.9 | 175.4 | 176.65 | 96.9 | 0.116 | 0.1825 | |
| +B2m | 0.25 | 180.9 | 177.9 | 179.4 | 99.65 | 0.119 | 0.1877 | |
| | NSB | 79.7 | 79.8 | 79.75 | 0 | | | |

With HIVgag competitor peptide

| | | | | | | | | |
|---|---|---|---|---|---|---|---|---|
| With | | | | | | | | |
| Competitor | | | | | | | | |
| 100X | | | | | | | | |
| −B2M | 0.25 | 84.2 | 85.2 | 84.7 | 4.95 | 0.005 | 0.0074 | 95.83 |
| +B2m | 0.25 | 84.7 | 85.4 | 85.05 | 5.3 | 0.005 | 0.0081 | 95.69 |
| | NSB | 79.7 | 79.8 | 79.75 | 0 | | | |
| No | | | | | | | | |
| Competitor | | | | | | | | |
| no Exchange | 0.25 | 172.5 | 176.4 | 174.45 | 94.7 | 0.1129 | 0.1783 | |
| +B2m | 0.25 | 178.7 | 180.2 | 179.45 | 99.7 | 0.1189 | 0.1878 | |
| | NSB | 79.7 | 79.8 | 79.75 | 0 | | | |

With EBV Bmlf-1 competitor peptide

| | | | | | | | | % |
|---|---|---|---|---|---|---|---|---|
| | | | | | | | | Fluorescent |
| | [monomer] | | | | NSB | Conc. | B/T | Monomer |
| | μg/ml | Bound 1 | Bound 2 | Mean | subtract | mg/ml | correction | vs 100% |
| With | | | | | | | | |
| Competitor | | | | | | | | |
| 100X | | | | | | | | |
| −B2M | 0.25 | 95 | 94.6 | 94.8 | 15.05 | 0.017 | 0.0267 | 84.88 |
| +B2m | 0.25 | 94.8 | 94.6 | 94.7 | 14.95 | 0.017 | 0.0265 | 85.33 |
| | NSB | 79.7 | 79.8 | 79.75 | 0 | | | |
| No | | | | | | | | |
| Competitor | | | | | | | | |
| −B2M | 0.25 | 173.1 | 173.8 | 173.45 | 93.7 | 0.112 | 0.1764 | |
| +B2m | 0.25 | 175.6 | 175.6 | 175.6 | 95.85 | 0.114 | 0.1805 | |
| | NSB | 79.7 | 79.8 | 79.75 | 0 | | | |

TABLE 12
Quantification of the total monomer.

| Monomer 26-35 + HBVc- | Exchanged | | | | | |
|---|---|---|---|---|---|---|
| FITC Peptide | peptide | Bound1 | Bound2 | Mean | Mean-NSB | [C] μg/ml |
| HIVpol peptide | 100 x | 54.8 | 47.4 | 51.1 | 45.4 | 0.137 |
| HIVpol peptide | 100X + b2m | 48.2 | 45.6 | 46.9 | 46.9 | 0.141 |
| Without competitor | 0 | 50.2 | 47.5 | 48.85 | 48.85 | 0.147 |
| Without competitor | 0 + b2m | 51.6 | 52.1 | 51.85 | 51.85 | 0.156 |
| CMVpp65 Peptide | 100 x | 53 | 51.6 | 52.3 | 52.3 | 0.157 |
| CMVpp65 peptide | 100X + b2m | 54.8 | 54.7 | 54.75 | 54.75 | 0.165 |
| Without competitor | 0 | 59.1 | 59.4 | 59.25 | 59.25 | 0.178 |
| Without competitor | 0 + b2m | 58.4 | 57.3 | 57.85 | 57.85 | 0.174 |
| HIVgag peptide | 100 x | 52.2 | 44.5 | 48.35 | 48.35 | 0.145 |
| HIVgag peptide | 100X + b2m | 44 | 38.4 | 41.2 | 41.2 | 0.124 |
| Without competitor | 0 | 49.6 | 52.3 | 50.95 | 50.95 | 0.153 |
| Without competitor | 0 + b2m | 53 | 54 | 53.5 | 53.5 | 0.161 |
| EBV BMLF1 peptide | 100 x | 52.6 | 47.6 | 50.1 | 50.1 | 0.151 |
| EBV BMLF1 peptide | 100X + b2m | 49.5 | 47.1 | 48.3 | 48.3 | 0.145 |
| Without competitor | 0 | 53.1 | 48 | 50.55 | 50.55 | 0.152 |
| Without comptetitor | 0 + b2m | 56.6 | 48.7 | 52.65 | 52.65 | 0.158 |

TABLE 13
Comparison of the monomer concentration calculated
with the HBVC-FITC and the B1G6-PE assay.

| | B1G6 | HBVc-FITC |
|---|---|---|
| Mean | 0.1527 | 0.182 |
| SD | 0.01349 | 0.0047 |
| CV | 8.83 | 2.61 |
| n | 16 | 8 |

TABLE 14
Flow cytometry results.

| | N9V Cells | | |
|---|---|---|---|
| | | | % of |
| | % Cells | MFI FL2 | Control |
| SA-PE | 100 | 0.286 | |
| CD3e-PE | 99 | 21.6 | |
| Tetramer with | | | |
| Variable [Monomer] | | | |
| Constant [SA-PE] | | | |
| in % [Monomer] | | | |
| 100 | 98.8 | 0.857 | 100.00 |
| 90 | 98.8 | 0.845 | 98.60 |
| 80 | 98.1 | 0.677 | 79.00 |
| 70 | 99.8 | 0.532 | 62.08 |
| 60 | 99.9 | 0.322 | 37.57 |

### EXAMPLE 4

Staining of PBMCs from HLA-A*0201/CMV Positives Donors with Tetramers Manufactured with Exchanged and Folded Monomers.

Identification of HLA-A*0201/CMVpp65 N9V positive healthy donors, allowed testing of tetramers manufactured with the monomer 26-35 exchanged with the CMVpp65 N9V peptide and comparison of the staining profile with the tetramer manufactured with the monomer folded with the N9V peptide. Results of the staining showed that the monomer used to stain these cells was exchanged at 90.38%. In this experiments we included the tetramer HLA-A*0201/Mart-1 26-35L as negative control. The staining was a triple immunostaining using an anti-CD3-PC5/CD8-FITC and the different tetramers with the SA-PE. The analysis was carried out taking into account the events located in the lymphocyte gate (FSC/SSC) combined with the CD3 positive and CD8 positive cells.

Although the invention has been described with reference to the above example, it will be understood that modifications and variations are encompassed within the spirit and scope of the invention. Accordingly, the invention is limited only by the following claims.

1. A method for identifying an MHC-binding peptide for an MHC monomer, or modified MHC monomer, said method comprising:
a) incubating under suitable liquid phase conditions a sample comprising:
at least one MHC monomer or modified MHC monomer having bound thereto a template MHC-binding peptide,
an excess amount of a first competitor peptide, and
a tracer MHC-binding peptide tagged with a detectable label so as to allow competition between the first competitor peptide, the template peptide, and the tracer peptide for binding to the MHC monomer or modified MHC monomer, wherein the template peptide has lower or intermediate affinity as compared with the tracer peptide for the monomer; and
b) determining a difference in signal produced by the detectable label in the sample as compared with signal produced solely by monomer obtained from the sample after the incubation, wherein the difference indicates the first competitor peptide is an MHC-binding peptide for the monomer.

2. The method of claim 1, wherein the excess of the first competitor peptide is about 100-fold molar excess.

3. The method of claim 1, wherein the tracer peptide displaces at least 90% of the template peptide in a parallel competition assay conducted in the absence of the first competitor peptide.

4. The method of claim 1, wherein the suitable liquid phase conditions include incubating the sample for about 2 to 20 hours.

5. The method of claim 4, wherein the suitable liquid phase conditions further include incubating the sample at about 21° C.

6. The method of claim 1, wherein the detectable label is a fluorophore.

7. The method of claim 6, wherein the monomer is attached to a solid support prior to determining the signal produced solely by the monomers in the sample.

8. The method of claim 7, wherein the MHC monomer or modified MHC monomer is biotinylated and the monomer is attached to the solid support via a biotin/avidin or streptavidin linkage.

9. The method of claim 6, wherein the fluorophore is fluorescein (FITC).

10. The method of claim 1, wherein the monomer is in a ternary complex further comprising beta-2 microglobulin.

11. The method of claim 1, wherein the difference is a decrease in the signal and binding of the first competitor peptide to the monomer is proportional to the amount of the decrease.

12. The method of claim 1, wherein the monomer is HLA class I.

13. The method of claim 12, wherein the monomer is HLA-A*020/Mart-1 26-35.

14. The method of claim 13, wherein the tracer peptide is HBc 18-27.

15. The method of claim 12, wherein the monomer is obtained from the sample in b) by cytometry.

16. The method of claim 1, wherein the method is repeated, except that a different competitor peptide is used.

17. The method of claim 1, wherein the determining comprises reading fluorescence using high throughput scanning.

18. The method of claim 1, wherein the monomer is HLA class I and monomer further binds with beta-2 microglobulin.

19. The method of claim 18, wherein the monomers is HLA subclass A, B or C.

20. The method of claim 18, wherein the first competitor peptide comprises from about 8 to about 12 amino acids.

21. The method of claim 20, wherein the monomer remains folded during the assay.

22. The method of claim 20, wherein the tracer peptide comprises from about 8 to about 12 amino acids and peptide exchange occurs without unfolding or denaturing of the monomer.

23. The method of claim 1, wherein the affinity of an exchanged competitor peptide is substantially equal to affinity of the first competitor peptide when folded into the binding pocket of the monomer during reconstitution of a ternary complex comprising the first competitor peptide and the monomer.

24. The method of claim 1, wherein the modified MHC monomer comprises cell surface domains of the MHC monomer but does not comprise other domains of the MHC monomer.

25. The method of claim 1, wherein the allele of the monomer is known and the determining indicates whether the first competitor peptide is specific for the allele of the monomer.

26. A method for measuring relative affinity of MHC-binding peptides for an MHC monomer, or modified MHC monomer, said method comprising:
a) incubating under suitable liquid phase conditions a sample comprising:
at least one MHC monomer or modified MHC monomer having bound thereto a template MHC-binding peptide,
an excess amount of a first competitor peptide, and
a tracer MHC-binding peptide tagged with a detectable label
so as to allow competition between the first competitor peptide, the template peptide, and the tracer peptide for binding to the MHC monomer or modified MHC monomer, wherein the template peptide has lower affinity than the tracer peptide for the monomer; and wherein at least a portion of the first competitor peptide exchanges with the template peptide; and
b) determining a difference in signal produced by the detectable label in the total sample as compared with signal produced solely by monomer obtained from the sample after the incubation, wherein the difference indicates affinity of the first competitor peptide for the monomer.

27. The method of claim 26, wherein the excess of the first competitor peptide is about 100-fold molar excess.

28. The method of claim 26, wherein the tracer peptide displaces at least 90% of the template peptide in a competition peptide exchange assay conducted in the absence of the first competitor peptide.

29. The method of claim 26, wherein the suitable liquid phase conditions include incubating the sample for about 2 to about 6 hours.

30. The method of claim 26, wherein the suitable liquid phase conditions further include incubating the sample at about 21° C.

31. The method of claim 26, wherein the detectable label is a fluorophore.

32. The method of claim 31, wherein the monomer is attached to a solid support prior to determining the signal produced solely by the monomers in the sample.

33. The method of claim 32, wherein the MHC monomer or modified MHC monomer is biotinylated and the monomer is attached to the solid support via a biotin/avidin or streptavidin linkage.

34. The method of claim 31, wherein the fluorophore is fluorescein (FITC).

35. The method of claim 26, wherein the monomer is in ternary complex further comprises beta-2 microglobulin.

36. The method of claim 26, wherein the difference is a decrease in the signal and binding of the first competitor peptide to the monomer is proportional to the amount of the decrease.

37. The method of claim 26, wherein the monomer is HLA class I.

38. The method of claim 37, wherein the monomer is HLA-A*020/Mart-1 27-35.

39. The method of claim 26, further comprising obtaining the monomer from the sample in b) by cytometry.

40. The method of claim 26, wherein the method is repeated, except that a different competitor peptide is used.

41. The method of claim 40, wherein the tracer peptide is HBc 18-27.

42. The method of claim 26, wherein the determining comprises reading fluorescence using high throughput scanning.

43. The method of claim 26, wherein the monomer is HLA class I and monomer further binds with beta-2 microglobulin.

44. The method of claim 43, wherein the monomer is HLA subclass A, B or C.

45. The method of claim 43, wherein the first competitor peptide comprises from about 8 to about 12 amino acids.

46. The method of claim 45, wherein peptide exchange occurs without unfolding or denaturing of the monomer.

47. The method of claim 45, wherein the tracer peptide comprises from about 8 to about 12 amino acids and peptide exchange occurs without unfolding or denaturing of the monomer.

48. The method of claim 26, wherein the affinity of a exchanged competitor peptide is substantially the equal to affinity of the first competitor peptide when folded into the binding pocket of the monomer during reconstitution of a ternary complex comprising the first competitor peptide and the monomer.

49. The method of claim 26, wherein the modified MHC monomer comprises cell surface domains of the MHC monomer but does not comprise other domains of the MHC monomer.

50. The method of claim 26, wherein the allele of the monomers is known and the determining determines whether the first competitor peptide is specific for the allele of the monomer.

51. A method for measuring function of an MHC-monomer or modified MHC monomer bound to an exchanged peptide for staining a cell displaying a peptide-restricted T-cell receptor (TCR), said method comprising:
a) incubating under suitable liquid phase conditions a sample comprising:
MHC monomers or modified MHC monomers having bound thereto a template MHC-binding peptide,
an excess amount of a first competitor peptide, and
a tracer MHC-binding peptide tagged with a first detectable label
so as to allow competition between the first competitor peptide, the template peptide and the tracer peptide for binding to the MHC monomer or modified MHC monomer, wherein the template peptide has lower affinity than the tracer peptide for the monomer and wherein, in at least a portion of the monomers, the first competitor peptide exchanges with the template peptide to form exchanged monomers;
b) forming a multimer of the exchanged monomers obtained from a) by binding the exchanged monomers with a multivalent entity labeled with a second detectable label; and
c) determining binding of the exchanged monomers in the multimer with the TCR of the cell, wherein the binding indicates the first competitor peptide in the exchanged monomers is specific for the TCR.

52. The method of claim 51, wherein the MHC monomer or modified MHC monomer is biotinylated and the multivalent entity is streptavidin or avidin.

53. The method of claim 51, wherein the first detectable label is FITC and the second detectable label is PE.

54. The method of claim 51, wherein the excess is about 100-fold molar excess.

55. The method of claim 51, wherein the tracer peptide displaces at least 90% of the template peptide in a competition peptide exchange assay conducted in the absence of the first competitor peptide.

56. The method of claim 51, wherein the suitable liquid phase conditions include incubating the sample for about 6 to about 20 hours.

57. The method of claim 56, wherein the suitable liquid phase conditions further include incubating the sample at about 21° C.

58. The method of claim 51, wherein the monomer is a ternary complex further comprising beta-2 microglobulin.

59. The method of claim 51, wherein the monomer is HLA class I.

60. The method of claim 59, wherein the monomer is HLA-A*020.

61. The method of claim 60, wherein the template peptide is Mart-1 26-35.

62. The method of claim 61, wherein the tracer peptide is HBc 18-27.

63. The method of claim 51, wherein the determining comprises reading fluorescence of the cells using high throughput scanning.

64. The method of claim 51, wherein the monomer is HLA class I and the monomer further comprises beta-2 microglobulin.

65. The method of claim 64, wherein the monomers is HLA subclass A, B or C.

66. The method of claim 51, wherein the first competitor peptide comprises from about 8 to about 12 amino acids.

67. The method of claim 66, wherein the tracer peptide comprises from about 8 to about 12 amino acids and peptide exchange occurs unfolding or denaturing of the monomer.

68. The method of claim 51, wherein peptide exchange occurs without unfolding or denaturing of the monomer.

69. The method of claim 51, wherein the binding of the multimer of exchanged monomers with the TCR is substantially equal to binding of a multimer prepared from the monomers in which the first competitor peptide is folded into the binding pocket of the monomer during reconstitution of a ternary complex.

70. The method of claim 51, wherein the modified MHC monomer comprises cell surface domains of the MHC monomer but does not comprise other domains of the MHC monomer.

71. The method of claim 51, wherein the peptide specificity of the TCR is known and the binding of the monomers in the multimer to the TCR indicates the exchanged monomer matches the peptide specificity of the TCR.

72. The method of claim 51, wherein the method is repeated except that a different competitor peptide is used in place of the first competitor peptide.

73. A system for identifying an MHC-binding peptide for an MHC monomer, or modified MHC monomer, said system comprising:
a) at least one MHC monomer or modified MHC monomer having bound thereto a template MHC-binding peptide,
b) a tracer MHC-binding peptide tagged with a detectable label wherein the template peptide has lower affinity than the tracer peptide for the monomer, and

74. The system of claim 73, wherein the system further comprises an instruction for using the system.

75. The system of claim 73, wherein the monomer is HLA-A*020 and the template peptide is Mart-1 26-35.

76. The system of claim 73, wherein the tracer peptide is HBc 18-27.

77. The system of claim 73, wherein the detectable label is FITC.

78. The system of claim 73, wherein the monomer is biotinylated for attachment to an avidinated solid support.
